(19) United States
(12) Patent Application Publication
(10) Pub. No.: US 2022/0000737 A1
(43) Pub. Date: Jan. 6, 2022

(54) COMPOSITION COMPRISING AT LEAST TWO ANIONIC SURFACTANTS, A NONIONIC SURFACTANT, AN AMPHOTERIC SURFACTANT, AND AT LEAST ONE DIRECT DYE

(71) Applicant: L'OREAL, Paris (FR)

(72) Inventors: Estelle MATHONNEAU, Saint Ouen (FR); Grégory PLOS, Chevilly La Rue (FR); Marie-Florence D'ARRAS, Saint Ouen (FR); Henri SAMAIN, Chevilly La Rue (FR)

(21) Appl. No.: 16/467,592

(22) PCT Filed: Dec. 15, 2017
(86) PCT No.: PCT/EP2017/082986

§ 371 Date: Jun. 7, 2019

(30) Foreign Application Priority Data
Dec. 16, 2016 (FR) 1662585

(51) Int. Cl. A61K 8/42; A61K 8/86; A61K 8/60; A61K 8/37; A61K 8/40; A61K 8/49; A61K 8/35; A61K 8/41; A61K 8/81; A61Q 5/10; A61Q 5/02

(52) U.S. Cl. CPC A61K 8/42; A61K 2800/432; A61K 8/602; A61K 8/375; A61K 8/40; A61K 8/494; A61K 8/355; A61K 8/418; A61K 8/416; A61K 8/4926; A61K 8/492; A61K 8/8152; A61Q 5/10; A61Q 5/02; A61K 8/86

(57) ABSTRACT

Composition comprising at least two anionic surfactants, a nonionic surfactant, an amphoteric surfactant, and at least one direct dye The present invention relates to a composition for the cosmetic treatment of keratin fibres, in particular of the hair, comprising at least two anionic surfactants, at least one nonionic surfactant, at least one amphoteric surfactant and at least one direct dye. The invention also relates to a cosmetic process for treating keratin fibres, especially for dyeing the hair, using such a composition, and also to a use of said composition.

The present invention relates to a composition for the cosmetic treatment of keratin fibres, in particular of human keratin fibres such as the hair, comprising at least two different anionic surfactants, at least one nonionic surfactant, at least one amphoteric surfactant and at least one direct dye. The invention also relates to a cosmetic process for treating keratin fibres using such a composition and also to a use of said composition.

It is known practice to dye keratin fibres by direct dyeing or semi-permanent dyeing. Direct dyeing or semi-permanent dyeing consists in introducing the colour via a coloured molecule which becomes adsorbed at the surface of the individual hair or which penetrates into the individual hair. Thus, the process conventionally used in direct dyeing consists in applying to keratin fibres direct dyes, which are coloured and colouring molecules that have affinity for the fibres, leaving the fibres in contact with the colouring molecules and then rinsing the fibres. Generally, this technique leads to chromatic colourings.

These dyeing hair compositions do, admittedly, have good dyeing power, but the cosmetic properties thereby imparted still remain to be improved, especially when they are applied to sensitized hair, i.e. hair that is generally damaged or embrittled by the action of external atmospheric agents, such as light and bad weather, and/or mechanical or chemical treatments, such as brushing, combing, dyeing, bleaching, permanent waving and/or relaxing.

Thus, it is often common practice to resort to care compositions using complementary cosmetic agents known as conditioning agents in order to improve the cosmetic properties of sensitized hair. These conditioning agents may, of course, also improve the cosmetic behaviour of natural hair.

However, these hair dyeing compositions are not necessarily entirely satisfactory and can still be improved, especially as regards the deposition of direct dyes onto keratin fibres.

Moreover, the hair compositions of the prior art are not entirely satisfactory either as regards the working qualities (speed of start of foaming and foam abundance, but also easy distribution of the composition and good rinseability) or as regards the persistence with respect to various external agents (for example shampoos, light, pollution).

There is thus a real need to develop hair dye compositions which have good dyeing properties, which are also capable of cleansing and/or conditioning keratin fibres and of giving the hair improved cosmetic properties, after one or more applications, without making the head of hair charged or lank, while at the same time maintaining good washing power, especially good foaming power (abundant foam, generated rapidly) and satisfactory working qualities (ease of spreading of the composition on the hair, especially on wet hair, and good rinseability).

This aim is achieved by the present invention, one subject of which is especially a cosmetic composition, preferably a hair composition, which is especially intended for the cosmetic treatment of keratin fibres, in particular of human keratin fibres such as the hair, comprising:

(i) one or more anionic surfactants chosen from polyoxyalkylenated alkyl(amido)ether carboxylic acids and/or salts thereof;

(ii) one or more anionic surfactants other than the polyoxyalkylenated alkyl(amido)ether carboxylic acid anionic surfactants (i);

(iii) one or more nonionic surfactants;

(iv) one or more amphoteric surfactants; and

(v) one or more direct dyes.

It was found that the composition according to the invention has satisfactory foaming power. It allows the production of an abundant, rapidly-generated foam, which spreads easily on keratin fibres and is easy to remove on rinsing.

Certain users of dye compositions may have more or less sensitized hair, i.e. hair that is damaged or embrittled by the action of external atmospheric agents such as light and bad weather, and/or the action of mechanical or chemical treatments such as brushing, combing, dyeing, bleaching, permanent-waving and/or relaxing. Thus, the composition according to the invention also makes it possible to improve the cosmetic properties imparted to the keratin fibres, especially to the hair, preferably sensitized hair. In particular, the composition according to the invention makes it possible to improve the disentangling, the suppleness and also the feel of the hair, without a build-up effect.

Moreover, the compositions according to the invention have a sparingly aggressive nature, since their application to the hair fibre in the long run causes little damage associated in particular with the gradual removal of the lipids or proteins contained in or at the surface of said fibre.

In addition, the composition according to the invention allows good deposition, or even good penetration, of direct dyes on/in keratin fibres and thus makes it possible to obtain improved dyeing action.

Furthermore, the composition according to the invention may impart cosmetic properties that are shampoo-resistant.

The composition according to the invention also has the advantage of being stable on storage both at room temperature (20-25° C.) and at 45° C., especially as regards its visual aspect and/or its viscosity.

For the purposes of the present invention, the term “stable” refers to a composition which, after two months of storage, shows no change in appearance, colour, odour, pH or viscosity.

In the text hereinbelow, and unless otherwise indicated, the limits of a range of values are included within that range, especially in the expressions “between” and “ranging from . . . to . . . ”.

Moreover, the expression “at least one” used in the present description is equivalent to the expression “one or more”, and may be replaced therewith.

Anionic Surfactants

As indicated above, the composition according to the invention comprises at least one anionic surfactant of polyoxyalkylenated alkyl(amido)ether carboxylic acid type (i) and at least one anionic surfactant (ii) other than the anionic surfactant(s) (i).

Thus, for the purposes of the invention, the cosmetic composition comprises at least two different anionic surfactants.

The term “anionic surfactant” means a surfactant comprising, as ionic or ionizable groups, only anionic groups.

In the present description, a species is termed “anionic” when it bears at least one permanent negative charge or when it can be ionized into a negatively charged species, under the conditions of use of the composition of the invention (for example the medium or the pH) and not comprising any cationic charge.

Polyoxyalkylenated Alkyl(amido)ether Carboxylic Acids (i)

The composition of the invention contains at least one anionic surfactant chosen from polyoxyalkylenated alkyl(amido)ether carboxylic acids and salts thereof, in particular those comprising from 2 to 50 alkylene oxide and in particular ethylene oxide groups.

The polyoxyalkylenatedalkyl(amido)ether carboxylic acids that may be used are preferably chosen from those of formula (1):

R₁ [symbol] (OC₂H₄)ₙ—OCH₂COOA (1)

in which
R1 represents a linear or branched C6-C24 alkyl or alkenyl radical, an alkyl(C8-C9)phenyl radical, a radical R2CONH—CH₂—CH₂— with R2 denoting a linear or branched C9-C21 alkyl or alkenyl radical,

preferably R1 is a C8-C20 and preferably C8-C18 alkyl radical,
n is an integer or decimal number (average value) ranging from 2 to 24 and preferably from 2 to 10,
A denotes H, ammonium, Na, K, Li, Mg, Ca or a monoethanolamine or triethanolamine residue.

It is also possible to use mixtures of compounds of formula (1), in particular mixtures of compounds containing different groups R1.

The polyoxyalkylenated alkyl(amido)ether carboxylic acids that are particularly preferred are those of formula (1) in which:
R1 denotes a C12-C14 alkyl, cocoyl, oleyl, nonylphenyl or octylphenyl radical,
A denotes a hydrogen or sodium atom, and
n ranges from 2 to 20, preferably from 2 to 10.

Even more preferentially, use is made of compounds of formula (1) in which R1 denotes a C12 alkyl radical, A denotes a hydrogen or sodium atom and n ranges from 2 to 10.

Use is preferably made of polyoxyalkylenated (C6-C24)alkyl ether carboxylic acids and salts thereof, polyoxyalkylenated (C6-C24)alkylamido ether carboxylic acids, in particular those comprising from 2 to 15 alkylene oxide groups, salts thereof, and mixtures thereof.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Among the commercial products that may preferably be used are the products sold by the company KAO under the names:

Akypo® NP 70 (R₁=nonylphenyl, n=7, A=H)

Akypo® NP 40 (R₁=nonylphenyl, n=4, A=H)

Akypo® OP 40 (R₁=octylphenyl, n=4, A=H)

Akypo® OP 80 (R₁=octylphenyl, n=8, A=H)

Akypo® OP 190 (R₁=octylphenyl, n=19, A=H)

Akypo® RLM 38 (R₁=(C₁₂-C₁₄)alkyl, n=4, A=H)

Akypo® RLM 38 NV (R₁=(C₁₂-C₁₄)alkyl, n=4, A=Na)

Akypo® RLM 45 CA (R₁=(C₁₂-C₁₄)alkyl, n=4.5, A=H)

Akypo® RLM 45 NV (R₁=(C₁₂-C₁₄)alkyl, n=4.5, A=Na)

Akypo® RLM 100 (R₁=(C₁₂-C₁₄)alkyl, n=10, A=H)

Akypo® RLM 100 NV (R₁=(C₁₂-C₁₄)alkyl, n=10, A=Na)

Akypo® RLM 130 (R₁=(C₁₂-C₁₄)alkyl, n=13, A=H)

Akypo® RLM 160 NV (R₁=(C₁₂-C₁₄)alkyl, n=16, A=Na)

or by the company Sandoz under the names:

Sandopan DTC-Acid (R₁=(C₁₃)alkyl, n=6, A=H)

Sandopan DTC (R₁=(C₁₃)alkyl, n=6, A=Na)

Sandopan LS 24 (R₁=(C₁₂-C₁₄)alkyl, n=12, A=Na)

Sandopan JA 36 (R₁=(C₁₃)alkyl, n=18, A=H),

and more particularly the products sold under the following names:

Akypo® RLM 45 (INCI: Laureth-5 carboxylic acid)

Akypo® RLM 100

Akypo® RLM 38.

The composition according to the invention preferably comprises said polyoxyalkylenated alkyl(amido)ether carboxylic acid(s) and/or salts thereof in a total amount ranging from 0.05% to 30% by weight, preferably from 0.1% to 25% by weight, better still from 0.5% to 20% by weight and preferentially from 1% to 10% by weight, relative to the total weight of the composition.

Additional Anionic Surfactants (ii)

As indicated above, the composition according to the invention comprises at least one additional anionic surfactant other than the polyoxyalkylenated alkyl(amido)ether carboxylic acids and/or salts thereof (i) described above.

Preferably, the additional anionic surfactants (ii) used in the composition according to the invention are chosen from anionic surfactants comprising in their structure one or more sulfate and/or sulfonate and/or phosphate and/or carboxylate groups, and/or mixtures thereof, preferably sulfate groups.

The anionic surfactant(s) (ii) may be oxyethylenated and/or oxypropylenated. The total average number of ethylene oxide (EO) and/or propylene oxide (PO) groups may then range from 1 to 50 and especially from 1 to 10.

The carboxylic anionic surfactants that may be used thus comprise at least one carboxylic or carboxylate function.

They may be chosen from the following compounds: acylglycinates, acyllactylates, acylsarcosinates, acylglutamates; alkyl-D-galactoside uronic acids; and also the salts of these compounds;

the alkyl and/or acyl groups of these compounds comprising from 6 to 30 carbon atoms, especially from 12 to 28, better still from 14 to 24 or even from 16 to 22 carbon atoms; these compounds possibly being polyoxyalkylenated, especially polyoxyethylenated, and then preferably comprise from 1 to 50 ethylene oxide units, better still from 1 to 10 ethylene oxide units.

Use may also be made of the C6-C24 alkyl monoesters of polyglycoside-polycarboxylic acids, such as C6-C24 alkyl polyglycoside-citrates, C6-C24 alkyl polyglycoside-tartrates and C6-C24 alkyl polyglycoside-sulfosuccinates, and salts thereof.

Preferentially, the carboxylic anionic surfactants are chosen, alone or as a mixture, from:
acylglutamates, especially of C6-C24 or even C12-C20, such as stearoylglutamates, and in particular disodium stearoylglutamate;
acylsarcosinates, in particular of C6-C24 or even C12-C20, such as palmitoylsarcosinates, and in particular sodium palmitoylsarcosinate;
acyllactylates, in particular of C12-C28 or even C14-C24, such as behenoyllactylates, and in particular sodium behenoyllactylate;
C6-C24 and in particular C12-C20 acylglycinates;

in particular in the form of alkali metal or alkaline-earth metal, ammonium or amino alcohol salts.

The sulfonate anionic surfactants that may be used comprise at least one sulfonate function.

They may be chosen from the following compounds: alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds;

the alkyl groups of these compounds comprising from 6 to 30 carbon atoms, in particular from 12 to 28, better still from 14 to 24 or even from 16 to 22 carbon atoms; where the aryl group preferably denotes a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Preferentially, the sulfonate anionic surfactants are chosen, alone or as a mixture, from:
C₆-C₂₄ and in particular C₁₂-C₂₀ alkylsulfosuccinates, in particular laurylsulfosuccinates;
C₆-C₂₄ and in particular C₁₂-C₂₀ alkyl ether sulfosuccinates;
(C6-C24)acylisethionates and preferably (C12-C18)acylisethionates;

in particular in the form of alkali metal or alkaline-earth metal, ammonium or amino alcohol salts.

The sulfate anionic surfactants that may be used comprise at least one sulfate function.

They may be chosen from the following compounds: alkyl sulfates, alkyl ether sulfates, alkylamido ether sulfates, alkylaryl polyether sulfates, monoglyceride sulfates; and the salts of these compounds;

the alkyl groups of these compounds comprising from 6 to 30 carbon atoms, in particular from 12 to 28, better still from 14 to 24 or even from 16 to 22 carbon atoms; where the aryl group preferably denotes a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Preferentially, the sulfate anionic surfactants are chosen, alone or as a mixture, from:
alkyl sulfates, in particular of C6-C24 or even C12-C20,
alkyl ether sulfates, in particular of C6-C24 or even C12-C20, preferably comprising from 2 to 20 ethylene oxide units;

in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium or calcium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the additional anionic surfactants (ii) are chosen, alone or as a mixture, from:
C₆-C₂₄ and in particular C₁₂-C₂₀ alkyl sulfates;
C₆-C₂₄ and in particular C₁₂-C₂₀ alkyl ether sulfates; preferably comprising from 2 to 20 ethylene oxide units;
C₆-C₂₄ and in particular C₁₂-C₂₀ alkylsulfosuccinates, in particular laurylsulfosuccinates;
C₆-C₂₄ and in particular C₁₂-C₂₀ alkyl ether sulfosuccinates;
(C6-C24)acylisethionates and preferably (C12-018)acylisethionates;
C6-C24 and in particular C12-C20 acylsarcosinates; in particular palmitoylsarcosinates;
C6-C24 and in particular C12-C20 acylglutamates;
C6-C24 and in particular C12-C20 acylglycinates;

in particular in the form of alkali metal or alkaline-earth metal, ammonium or amino alcohol salts.

Among the anionic surfactants (ii), use is preferably made of one or more sulfate anionic surfactants, preferentially chosen from C8-C14 and more particularly C12-C14 alkyl sulfates and alkyl ether sulfates, and more particularly lauryl (ether) sulfates.

Preferably, the anionic surfactant(s) (ii) are in the form of salts, and in particular alkaline salts, especially sodium salts, ammonium salts, amine salts, including amino alcohol salts, and/or magnesium salts. These salts preferably comprise from 2 to 5 ethylene oxide groups.

Among these salts, sodium, triethanolamine, magnesium or ammonium (C₁₂-C₁₄)alkyl sulfates and/or sodium, ammonium or magnesium (C₁₂-C₁₄)alkyl ether sulfates, which are oxyethylenated, for example with 1 to 10 mol of ethylene oxide, are more preferably used.

Better still, the anionic surfactant(s) (ii) are chosen from sodium, ammonium or magnesium (C₁₂-C₁₄)alkyl ether sulfates oxyethylenated with 2.2 mol of ethylene oxide, as sold under the name Texapon N702 by the company Cognis.

The anionic surfactant(s) (ii) may be present in the composition according to the invention in a total content ranging from 0.05% to 30% by weight, preferably in a content ranging from 0.1% to 25% by weight and better still from 0.5% to 20% by weight, even better still from 1% to 15% by weight, relative to the total weight of the composition.

According to a particular embodiment, the total content of anionic surfactants (i) and (ii) in the composition according to the invention is between 0.1% and 35% by weight, preferably between 1% and 25% by weight, and preferentially between 5% and 15% by weight, relative to the total weight of the composition.

Nonionic Surfactants (iii)

The composition comprises one or more nonionic surfactants, chosen from:

(a) oxyethylenated alcohols comprising at least one saturated or unsaturated, linear or branched C8 to C40 alkyl chain, and comprising from 1 to 100 mol of ethylene oxide, preferably from 2 to 50 and more particularly from 2 to 40 mol of ethylene oxide and comprising one or two fatty chains;

(b) nonionic surfactants of alkyl(poly)glycoside type, represented especially by the following general formula: R1O—(R2O)t-(G)v in which:
R1 represents a linear or branched alkyl or alkenyl radical comprising 6 to 24 carbon atoms and especially 8 to 18 carbon atoms, or an alkylphenyl radical whose linear or branched alkyl radical comprises 6 to 24 carbon atoms and especially 8 to 18 carbon atoms,
R2 represents an alkylene radical comprising 2 to 4 carbon atoms,
G represents a sugar unit comprising 5 to 6 carbon atoms,
t denotes a value ranging from 0 to 10 and preferably 0 to 4,
v denotes a value ranging from 1 to 15 and preferably 1 to 4; and/or

(c) polyethoxylated fatty acid esters of sorbitan, preferably containing from 2 to 40 mol of ethylene oxide and comprising at least one saturated or unsaturated, linear or branched C8 to C40 alkyl chain, preferably C10-C28 alkyl chain (fatty acid);

(d) fatty acid esters of sucrose, preferably comprising at least one saturated or unsaturated, linear or branched 08 to 040 alkyl chain, preferably C10-C28 alkyl chain (fatty acid), such as sucrose cocoate and sucrose palmitate; and/or

(e) polyglycerolated fatty esters, the number of glycerol groups possibly ranging from 2 to 30 and comprising at least one saturated or unsaturated, linear or branched 08 to 040 alkyl chain, preferably C10-C28 alkyl chain (fatty acid), such as polyglyceryl-5 laurate, polyglyceryl-4 laurate, polyglyceryl-10 laurate, polyglyceryl-6 dicaprate.

Preferably, the alkyl(poly)glycoside surfactants are compounds of the formula described above in which:
R1 denotes a linear or branched, saturated or unsaturated alkyl radical comprising from 8 to 18 carbon atoms,
R2 represents an alkylene radical comprising 2 to 4 carbon atoms,
t denotes a value ranging from 0 to 3 and preferably equal to 0,
G denotes glucose, fructose or galactose, preferably glucose;
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C₈/C₁₆ alkyl(poly)glucosides 1,4, and in particular decyl glucosides and caprylyl/capryl glucosides, are most particularly preferred. Among commercial products, mention may be made of the products sold by the company Cognis under the names Plantaren® (600 CS/U, 1200 and 2000) or Plantacare® (818, 1200 and 2000); the products sold by the company SEPPIC under the names Oramix CG 110 and Oramix® NS 10; the products sold by the company BASF under the name Lutensol GD 70, or else the products sold by the company Chem Y under the name AG10 LK.

Preferably, use is made of C₈/C₁₆-alkyl (poly)glycosides 1,4, in particular as an aqueous 53% solution, such as those sold by Cognis under the reference Plantacare® 818 UP.

Preferentially, the nonionic surfactants are chosen, alone or as a mixture, from:
saturated or unsaturated, linear or branched, oxyethylenated C₈ to C₄₀, especially C₈-C₂₀ and better still C₁₀-C₁₈ fatty alcohols comprising from 1 to 100 mol of ethylene oxide, preferably from 2 to 50, more particularly from 2 to 40 mol, or even from 3 to 20 mol of ethylene oxide; especially lauryl alcohol containing 4 mol of ethylene oxide (INCI name: Laureth-4) and lauryl alcohol containing 12 mol of ethylene oxide (INCI name: Laureth-12); and
(C6-C24 alkyl)(poly)glycosides, and more particularly (C8-C18 alkyl)(poly)glycosides.

Better still, the nonionic surfactants are preferentially chosen from oxyethylenated alcohols comprising at least one C₈-C₂₀ and better still C₁₀-C₁₈ alkyl chain, comprising from 2 to 50 and in particular from 3 to 20 mol of ethylene oxide.

According to a preferred embodiment of the invention, the composition comprises at least two nonionic surfactants chosen from oxyethylenated alcohols comprising at least one C₈-C₂₀ and better still C₁₀-C₁₈ alkyl chain, comprising from 2 to 50 and in particular from 3 to 20 mol of ethylene oxide and (C₆-C₂₄ alkyl)(poly)glycosides, and more particularly (C₈-C₁₈ alkyl)(poly)glycosides.

Preferably, the nonionic surfactant(s) are present in the composition according to the invention in a total content ranging from 0.05% to 30% by weight, preferentially ranging from 0.1% to 25% by weight, in particular ranging from 0.5% to 20% by weight, especially from 1% to 15% by weight and better still from 2% to 10% by weight, relative to the total weight of the composition.

Amphoteric Surfactants (iv)

As indicated above, the composition comprises one or more amphoteric surfactants, preferably present in a total content of greater than or equal to 3% by weight relative to the total weight of the composition.

In particular, the amphoteric or zwitterionic surfactant(s) are non-silicone surfactants. They may especially be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines.

Among the optionally quaternized derivatives of secondary or tertiary aliphatic amines that may be used, as defined above, mention may also be made of the compounds having the respective structures (II) and (III) below:

Rₐ—CONHCH₂CH₂—N⁺(R_{b})(R_{c})—CH₂COO⁻,M⁺,X⁻ (II)

in which:
Rₐ represents a C₁₀ to C₃₀ alkyl or alkenyl group derived from an acid RₐCOOH preferably present in hydrolysed coconut oil, or a heptyl, nonyl or undecyl group;
R_{b} represents a beta-hydroxyethyl group; and
R_{c} represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

R_{a′}—CONHCH₂CH₂—N(B)(B′) (III)

in which:
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or —CH₂CH₂—COOZ′, or a hydrogen atom;
Y′ represents the group —COOH, —COOZ′ or —CH₂—CH(OH)SO₃H or the group CH₂CH(OH)SO₃—Z′;
Z′ represents a cationic counterion resulting from an alkali metal or alkaline-earth metal, such as sodium, an ammonium ion or an ion resulting from an organic amine;
R_{a′} represents a C₁₀ to C₃₀ alkyl or alkenyl group of an acid R_{a′}—COOH which is preferably present in coconut oil or in hydrolysed linseed oil, or an alkyl group, especially a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (IV):

R_{a″}—NHCH(Y″)—(CH₂)ₙCONH(CH₂)_{n″}—N(R_{d})(Rₑ) (IV)

in which:
Y″ represents the group —COOH, —COOZ″ or —CH₂—CH(OH)SO₃H or the group CH₂CH(OH)SO₃—Z″;
R_{d} and Rₑ represent, independently of each other, a C₁ to C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀ to C₃₀ alkyl or alkenyl group of an acid R_{a″}—COOH which is preferably present in coconut oil or in hydrolysed linseed oil;
n and n′ denote, independently of each other, an integer ranging from 1 to 3.

Mention may be made, among the compounds of formula (II), of the compound classified in the CTFA dictionary under the name sodium diethylaminopropyl cocoaspartamide and sold by Chimex under the name Chimexane HB.

These compounds may be used alone or as mixtures.

Among the amphoteric or zwitterionic surfactants, use is preferably made of (C₈-C₂₀)alkylbetaines such as cocoylbetaine, (C₈-C₂₀)alkylamido(C₃-C₈)alkylbetaines such as cocamidopropylbetaine, and mixtures thereof, and the compounds of formula (IV) such as the sodium salt of diethylaminopropyl laurylaminosuccinamate (INCI name: sodium diethylaminopropyl cocoaspartamide).

Preferentially, the amphoteric or zwitterionic surfactants are chosen from (C₈-C₂₀)alkylamido(C₃-C₈)alkylbetaines such as cocamidopropylbetaine.

Preferably, the amphoteric surfactant(s) are present in the composition according to the invention in a total content ranging from 0.1% to 25% by weight, preferentially in a content ranging from 1% to 20% by weight, especially from 3% to 15% by weight and better still from 3.5% to 10% by weight, relative to the total weight of the composition.

Direct Dyes

The direct dyes that may be used in the compositions according to the invention are preferably chosen, alone or as a mixture, from synthetic or natural, anionic, cationic or nonionic direct dyes.

Examples of suitable direct dyes that may be mentioned include azo direct dyes; (poly)methine dyes such as cyanines, hemicyanines and styryls; carbonyl dyes; azine dyes; nitro(hetero)aryl dyes; tri(hetero)arylmethane dyes; porphyrin dyes; phthalocyanine dyes and natural direct dyes, alone or in the form of mixtures.

Among the cationic direct dyes that may be used according to the invention, mention may be made of the hydrazono cationic dyes of formulae (IIIa) and (III′a), the azo cationic dyes (IVa) and (IV′a) and the diazo cationic dyes (Va) below:

| | | |
|---|---|---|
| Het⁺—C(R^{a})═N—N(R^{b})—Ar, An⁻ | Het⁺—N(R^{a})—N═C(R^{b})—Ar, An⁻ | Het⁺—N═N—Ar, An⁻ |
| (IIIa) | (III′a) | (IVa) |

Ar⁺—N═N—Ar″, An⁻ (IV′a) and
Het⁺—N═N—Ar′—N═N—Ar, An⁻ (Va)

in which:
Het⁺ represents a cationic heteroaryl radical, preferably bearing an endocyclic cationic charge, such as imidazolium, indolium or pyridinium, optionally substituted preferably with one or more (C₁-C₈)alkyl groups such as methyl;
Ar⁺ represents an aryl radical, such as phenyl or naphthyl, bearing an exocyclic cationic charge, preferably ammonium, particularly tri(C₁-C₈)alkylammonium such as trimethylammonium;
Ar represents an aryl group, especially phenyl, which is optionally substituted, preferably with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or, as a variant, Ar represents a julolidine group;
Ar′ represents an optionally substituted divalent (hetero)arylene group such as phenylene, particularly para-phenylene, or naphthalene, which is optionally substituted, preferably with one or more (C₁-C₈)alkyl, hydroxyl or (C₁-C₈)alkoxy groups;
Ar″ represents an optionally substituted (hetero)aryl group such as phenyl or pyrazolyl, which is optionally substituted, preferably with one or more (C₁-C₈)alkyl, hydroxyl, (di)(C₁-C₈)(alkyl)amino, (C₁-C₈)alkoxy or phenyl groups;
R^{a} and R^{b}, which may be identical or different, represent a hydrogen atom or a (C1-C8)alkyl group, which is optionally substituted, preferably with a hydroxyl group;

or, as a variant, the substituent R^{a} with a substituent of Het⁺ and/or R^{b} with a substituent of Ar and/or R^{a} with R^{b} form, together with the atoms that bear them, a (hetero)cycloalkyl;

particularly, R^{a} and R^{b} represent a hydrogen atom or a (C₁-C₄)alkyl group, which is optionally substituted with a hydroxyl group;
An⁻ represents an anionic counterion, such as mesylate or halide.

Mention may be made in particular of azo and hydrazono cationic dyes bearing an endocyclic cationic charge of formulae (IIIa), (III′a) and (IVa) as defined previously. More particularly those of formulae (IIIa), (III′a) and (IVa) derived from the dyes described in patent applications WO 95/15144, WO 95/01772 and EP-714954.

Preferably, the cationic part is derived from the following derivatives:

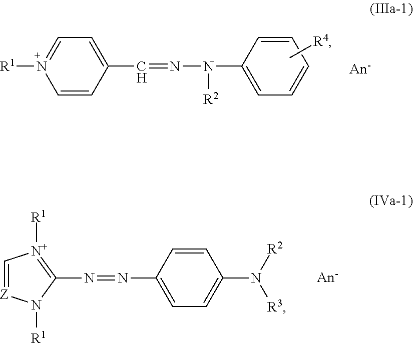

in which:
R¹, which may be identical different, represents a (C₁-C₄)alkyl group such as methyl;
R² and R³, which may be identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; in particular, R⁴ represents a hydrogen atom;
Z represents a CH group or a nitrogen atom, preferably CH;
An⁻ represents an anionic counterion, such as mesylate or halide.

In particular, the dye of formulae (IIIa-1) and (IVa-1) is chosen from Basic Red 51, Basic Yellow 87 and Basic Orange 31 or corresponding derivatives:

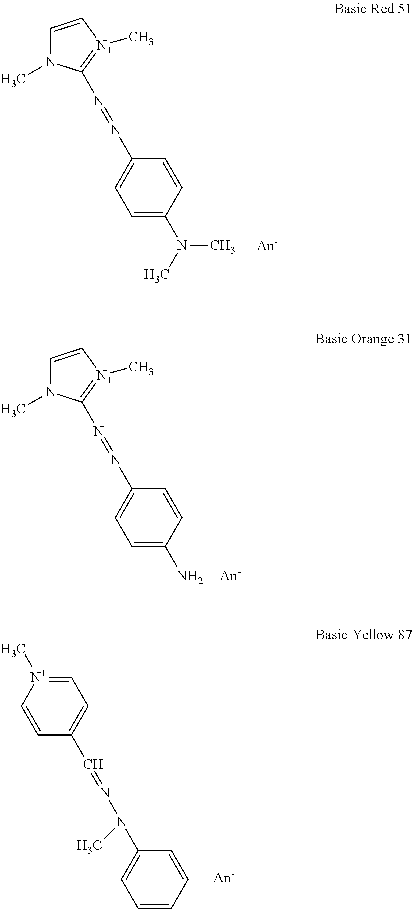

Among the natural direct dyes that may be used according to the invention, mention may be made of hennotannic acid, juglone, alizarin, purpurin, carminic acid, kermesic acid, purpurogallin, protocatechaldehyde, indigo, isatin, curcumin, spinulosin, apigenidin and orcein. Extracts or decoctions containing these natural dyes and in particular henna-based poultices or extracts may also be used.

Preferentially, the direct dyes according to the invention are chosen, alone or as a mixture, from anionic direct dyes.

The anionic direct dyes of the invention are dyes commonly referred to as “acid direct dyes” owing to their affinity for alkaline substances. The term “anionic direct dyes” means any direct dye comprising in its structure at least one CO₂R or SO₃R substituent with R denoting a hydrogen atom or a cation originating from a metal or an amine, or an ammonium ion. The anionic dyes may be chosen from direct nitro acid dyes, azo acid dyes, azine acid dyes, triarylmethane acid dyes, indoamine acid dyes, anthraquinone acid dyes, indigoid dyes and natural acid dyes.

As acid dyes according to the invention, mention may be made of the dyes of formulae (II), (II′), (III), (III′), (IV), (IV′), (V), (V′), (VI), (VII), (VIII) and (IX) below:

a) the diaryl anionic azo dyes of formula (II) or (II′):

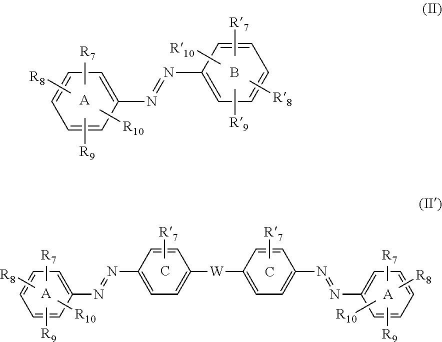

in which formulae (II) and (II′):
R₇, R₈, R₉, R₁₀, R′₇, R′₈, R′₉ and R′₁₀, which may be identical or different, represent a hydrogen atom or a group chosen from:
   alkyl;
   alkoxy, alkylthio;
   hydroxyl, mercapto;
   nitro, nitroso;
   R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
   R″—S(O)₂—, with R″ representing a hydrogen atom or an alkyl, aryl, (di)(alkyl)amino or aryl(alkyl)amino group; preferentially a phenylamino or phenyl group;
   R″—S(O)₂—X′— with R′″ representing an alkyl or optionally substituted aryl group, X′ as defined previously;
   (di)(alkyl)amino;
   aryl(alkyl)amino optionally substituted with one or more groups chosen from i) nitro; ii) nitroso; iii) (O)₂S(O⁻)—, M⁺ and iv) alkoxy with M⁺ as defined previously;
   optionally substituted heteroaryl; preferentially a benzothiazolyl group;
   cycloalkyl; especially cyclohexyl;
   Ar—N═N— with Ar representing an optionally substituted aryl group; preferentially a phenyl optionally substituted with one or more alkyl, (O)₂S(O⁻)—, M⁺ or phenylamino groups;
   or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{∘}—C(X)—X′—; viii) R^{∘}—X′—C(X)—; ix) R^{∘}—X′—C(X)—X″—; x) Ar—N═N— and xi) optionally substituted aryl(alkyl)amino; with M⁺, R^{∘}, X, X′, X″ and Ar as defined previously;
W represents a sigma bond, an oxygen or sulfur atom, or a divalent radical i) —NR— with R as defined previously, or ii) methylene —C(Rₐ)(R_{b})— with Rₐ and R_{b}, which may be identical or different, representing a hydrogen atom or an aryl group, or alternatively Rₐ and R_{b} form, with the carbon atom that bears them, a spiro cycloalkyl; preferentially, W represents a sulfur atom or Rₐ and R_{b} together form a cyclohexyl; it being understood that formulae (II) and (II′) comprise at least one sulfonate radical (O)₂S(O⁻)—, M⁺ or one carboxylate radical (O)CO⁻—, M⁺ on one of the rings A, A′, B, B′ or C; preferentially sodium sulfonate.

Among the dyes of formula (II) that may be mentioned are: Acid Red 1, Acid Red 4, Acid Red 13, Acid Red 14, Acid Red 18, Acid Red 27, Acid Red 28, Acid Red 32, Acid Red 33, Acid Red 35, Acid Red 37, Acid Red 40, Acid Red 41, Acid Red 42, Acid Red 44, Pigment red 57, Acid Red 68, Acid Red 73, Acid Red 135, Acid Red 138, Acid Red 184, Food Red 1, Food Red 13, Acid Orange 6, Acid Orange 7, Acid Orange 10, Acid Orange 19, Acid Orange 20, Acid Orange 24, Yellow 6, Acid Yellow 9, Acid Yellow 36, Acid Yellow 199, Food Yellow 3, Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Food yellow 3 or sunset yellow.

Among the dyes of formula (II′) that may be mentioned are: Acid Red 111, Acid Red 134, Acid Yellow 38;

b) the pyrazolone anionic azo dyes of formulae (III) and (III′):

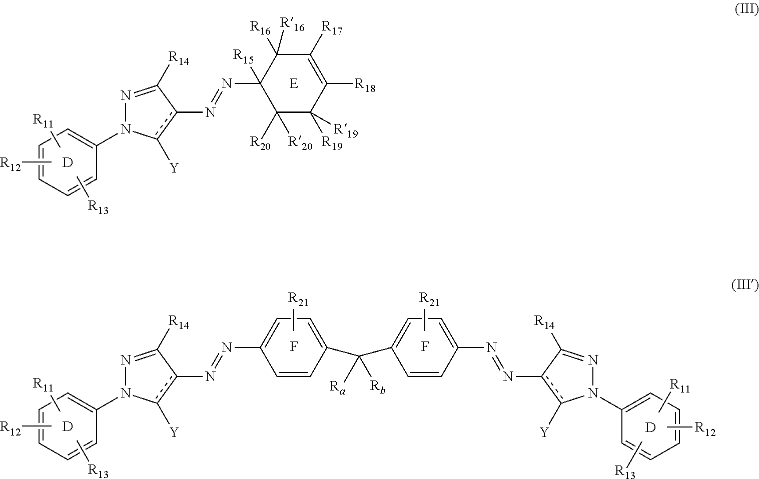

in which formulae (III) and (III′):
R₁₁, R₁₂ and R₁₃, which may be identical or different, represent a hydrogen or halogen atom, an alkyl group or —(O)₂S(O⁻), M⁺ with M⁺ as defined previously;
R₁₄ represents a hydrogen atom, an alkyl group or a group —C(O)O—, M⁺ with M⁺ as defined previously;
R₁₅ represents a hydrogen atom;
R₁₆ represents an oxo group, in which case R′₁₆ is absent, or alternatively R₁₅ with R₁₆ together form a double bond;
   R₁₇ and R₁₈, which may be identical or different, represent a hydrogen atom, a group chosen from i) (O)₂S(O⁻)—, M⁺ with M⁺ as defined previously, and ii) Ar—O—S(O)₂— with Ar representing an optionally substituted aryl group; preferentially a phenyl optionally substituted with one or more alkyl groups;
R₁₉ and R₂₀ together form either a double bond, or a benzo group D′, which is optionally substituted;
R′₁₆, R′₁₉ and R′₂₀, which may be identical or different, represent a hydrogen atom or an alkyl or hydroxyl group;
R₂₁ represents a hydrogen atom or an alkyl or alkoxy group;
Rₐ and Rₐ, which may be identical or different, are as defined previously, preferentially Rₐ represents a hydrogen atom and R_{b} represents an aryl group;
Y represents either a hydroxyl group or an oxo group;
[symbol] represents a single bond when Y is an oxo group; and represents a double bond when Y represents a hydroxyl group;

it being understood that formulae (III) and (III′) comprise at least one sulfonate radical (O)₂S(O⁻)—, M⁺ or one carboxylate radical C(O)O⁻—, M⁺ on one of the rings D or E; preferentially sodium sulfonate;

Among the dyes of formula (III) that may be mentioned are: Acid Red 195, Acid Yellow 23, Acid Yellow 27, Acid Yellow 76, and as examples of dyes of formula (III′), mention may be made of: Acid Yellow 17;

c) the anthraquinone dyes of formulae (IV) and (IV′):

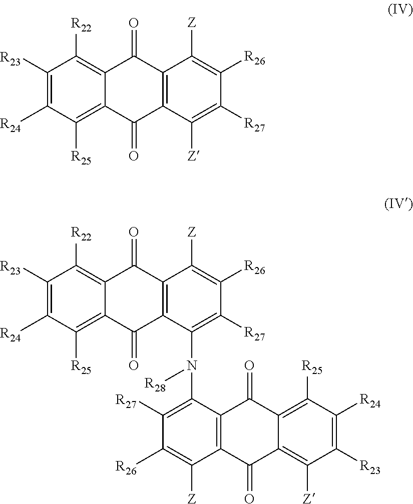

in which formulae (IV) and (IV′):
R₂₂, R₂₃, R₂₄, R₂₅, R₂₆ and R₂₇, which may be identical or different, represent a hydrogen or halogen atom, or a group chosen from:
   alkyl;
   hydroxyl, mercapto;
   alkoxy, alkylthio;
   optionally substituted aryloxy or arylthio, preferentially substituted with one or more groups chosen from alkyl and (O)₂S(O⁻)—, M⁺ with M⁺ as defined previously;
   aryl(alkyl)amino optionally substituted with one or more groups chosen from alkyl and (O)₂S(O⁻)—, M⁺ with M⁺ as defined previously;
   (di)(alkyl)amino;
   (di)(hydroxyalkyl)amino;
   (O)₂S(O⁻)—, M⁺ with M⁺ as defined previously;
Z′ represents a hydrogen atom or a group NR₂₈R₂₉ with R₂₈ and R₂₉, which may be identical or different, representing a hydrogen atom or a group chosen from:
   alkyl;
   polyhydroxyalkyl such as hydroxyethyl;
   aryl optionally substituted with one or more groups, particularly i) alkyl such as methyl, n-dodecyl, n-butyl; ii) (O)₂S(O⁻)—, M⁺ with M⁺ as defined previously; iii) R^{∘}—C(X)—X′—, R^{∘}—X′—C(X)—, R^{∘}—X′—C(X)—X″— with R^{∘}, X, X′ and X″ as defined previously, preferentially R^{∘} represents an alkyl group;
   cycloalkyl; especially cyclohexyl;
Z, represents a group chosen from hydroxyl and NR′₂₈R′₂₉ with R′₂₈ and R′₂₉, which may be identical or different, representing the same atoms or groups as R₂₈ and R₂₉ as defined previously;

it being understood that formulae (IV) and (IV′) comprise at least one sulfonate radical (O)₂S(O⁻)—, M⁺ or one carboxylate radical —C(O)O⁻—, M⁺; preferentially sodium sulfonate;

Among the dyes of formula (IV) that may be mentioned are: Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT violet No. 2; and, as an example of a dye of formula (IV′), mention may be made of: Acid Black 48;

d) the nitro dyes of formulae (V) and (V′):

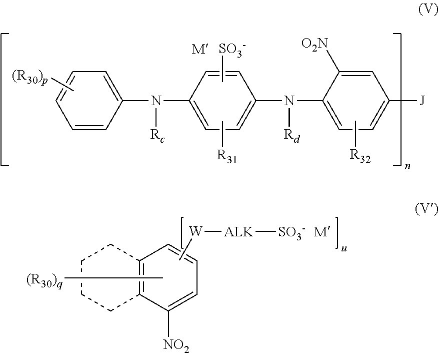

in which formulae (V) and (V′):
R₃₀, R₃₁ and R₃₂, which may be identical or different, represent a hydrogen or halogen atom, or a group chosen from:
   alkyl;
   alkoxy optionally substituted with one or more hydroxyl groups, alkylthio optionally substituted with one or more hydroxyl groups;
   hydroxyl, mercapto;
   nitro, nitroso;
   polyhaloalkyl;
   R^{∘}—C(X)—X′—, R^{∘}—X′—C(X)—, R^{∘}—X′—C(X)—X″— with R^{∘}, X, X′ and X″ as defined previously;
   (O)₂S(O⁻)—, M⁺ with M⁺ as defined previously;
   (O)CO⁻—, M⁺ with M⁺ as defined previously;
   (di)(alkyl)amino;
   (di)(hydroxyalkyl)amino;
   heterocycloalkyl such as piperidino, piperazino or morpholino;

in particular, R₃₀, R₃₁ and R₃₂ represent a hydrogen atom;
R_{c} and R_{d}, which may be identical or different, represent a hydrogen atom or an alkyl group;
W is as defined previously; W particularly represents a group —NH—;
ALK represents a linear or branched divalent C₁-C₆ alkylene group; in particular, ALK represents a group —CH₂—Cl₂—;
n is 1 or 2;
p represents an integer between 1 and 5 inclusive;
q represents an integer between 1 and 4 inclusive;
u is 0 or 1;
when n is 1, J represents a nitro or nitroso group; particularly nitro;
when n is 2, J represents an oxygen or sulfur atom, or a divalent radical —S(O)ₘ— with m representing an integer 1 or 2; preferentially, J represents a radical —SO₂—;
M′ represents a hydrogen atom or a cationic counterion;

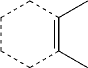

when it is present, represents a benzo group optionally substituted with one or more groups R₃₀ as defined previously;

it being understood that formulae (V) and (V′) comprise at least one sulfonate radical (O)₂S(O⁻)—, M⁺ or one carboxylate radical —C(O)O⁻—, M⁺; preferentially sodium sulfonate;

As dyes of formula (V), mention may be made of: Acid Brown 13 and Acid Orange 3; as examples of dyes of formula (V′), mention may be made of: Acid Yellow 1, the sodium salt of 2,4-dinitro-1-naphthol-7-sulfonic acid, 2-piperidino-5-nitrobenzenesulfonic acid, 2(4′-N,N(2″-hydroxyethyl)amino-2′-nitro)anilineethanesulfonic acid, 4-β-hydroxyethylamino-3-nitrobenzenesulfonic acid; EXT D&C Yellow 7;

e) the triarylmethane dyes of formula (VI):

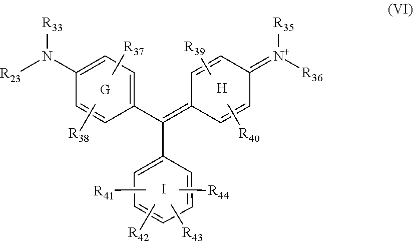

in which formula (VI):
R₃₃, R₃₄, R₃₅ and R₃₆, which may be identical or different, represent a hydrogen atom or a group chosen from alkyl, optionally substituted aryl and optionally substituted arylalkyl; particularly an alkyl and benzyl group optionally substituted with a group (O)ₘS(O⁻)—, M⁺ with M⁺ and m as defined previously;
R₃₇, R₃₈, R₃₉, R₄₀, R₄₁, R₄₂, R₄₃ and R₄₄, which may be identical or different, represent a hydrogen atom or a group chosen from:
   alkyl;
   alkoxy, alkylthio;
   (di)(alkyl)amino;
   hydroxyl, mercapto;
   nitro, nitroso;
   R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
   or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{∘}—C(X)—X′—; viii) R^{∘}—X′—C(X)— and ix) R^{∘}—X′—C(X)X″—; with M⁺, R^{∘}, X, X′ and X″ as defined previously;

particularly, R₃₇ to R₄₀ represent a hydrogen atom, and R₄₁ to R₄₄, which may be identical or different, represent a hydroxyl group or (O)₂S(O⁻)—, M⁺; and when R₄₃ with R₄₄ together form a benzo group, it is preferentially substituted with a group (O)₂S(O⁻) —;

it being understood that at least one of the rings G, H, I or I′ comprises at least one sulfonate radical (O)₂S(O⁻)— or a carboxylate radical —C(O)O⁻; preferentially sulfonate;

as examples of dyes of formula (VI), mention may be made of: Acid Blue 1; Acid Blue 3; Acid Blue 7, Acid Blue 9; Acid Violet 49; Acid Green 3; Acid Green 5 and Acid Green 50;

f) the xanthene-based dyes of formula (VII):

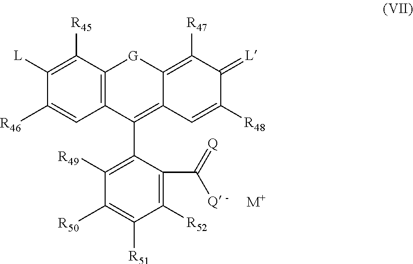

in which formula (VII):
R₄₅, R₄₆, R₄₇ and R₄₈, which may be identical or different, represent a hydrogen or halogen atom;
R₄₉, R₅₀, R₅₁ and R₅₂, which may be identical or different, represent a hydrogen or halogen atom, or a group chosen from:
   alkyl;
   alkoxy, alkylthio;
   hydroxyl, mercapto;
   nitro, nitroso;
   (O)₂S(O⁻)—, M⁺ with M⁺ representing a hydrogen atom or a cationic counterion;
   (O)CO⁻—, M⁺ with M⁺ as defined previously;

particularly, R53, R54, R55 and R45 represent a hydrogen or halogen atom;
G represents an oxygen or sulfur atom or a group NRₑ with Rₑ as defined previously; particularly G represents an oxygen atom;
L represents an alkoxide O⁻, M⁺; a thioalkoxide S⁻, M⁺ or a group NR_{f}, with R_{f} representing a hydrogen atom or an alkyl group and M⁺ as defined previously; M⁺ is particularly sodium or potassium;
L′ represents an oxygen or sulfur atom or an ammonium group: N⁺R_{f}R_{g}, with R_{f} and R_{g}, which may be identical or different, representing a hydrogen atom, an alkyl group or optionally substituted aryl; L′ represents more particularly an oxygen atom or a phenylamino group optionally substituted with one or more alkyl or (O)ₘS(O⁻)—, M⁺ groups with m and M⁺ as defined previously;
Q and Q′, which may be identical or different, represent an oxygen or sulfur atom; particularly, Q and Q′ represent an oxygen atom;
M⁺ is as defined previously;

as examples of dyes of formula (VI), mention may be made of: Acid Yellow 73; Acid Red 51; Acid Red 52; Acid Red 87; Acid Red 92; Acid Red 95; Acid Violet 9;

g) the indole-based dyes of formula (VIII):

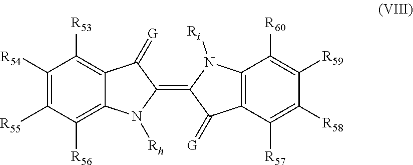

R₅₃, R₅₄, R₅₅, R₅₆, R₅₇, R₅₈, R₅₉ and R₆₀, which may be identical or different, represent a hydrogen atom or a group chosen from:
   alkyl;
   alkoxy, alkylthio;
   hydroxyl, mercapto;
   nitro, nitroso;
   R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, M⁺ with M⁺ representing a hydrogen atom or a cationic counterion;
   (O)CO⁻—, M⁺ with M⁺ as defined previously;
G represents an oxygen or sulfur atom or a group NRₑ with Rₑ as defined previously; particularly G represents an oxygen atom;
Rᵢ and Rₕ, which may be identical or different, represent a hydrogen atom or an alkyl group;

it being understood that formula (VIII) comprises at least one sulfonate radical (O)₂S(O⁻)—, M⁺ or one carboxylate radical —C(O)O⁻, M⁺; preferentially sodium sulfonate;

As examples of dyes of formula (VIII), mention may be made of: Acid Blue 74;

h) the quinoline-based dyes of formula (IX):

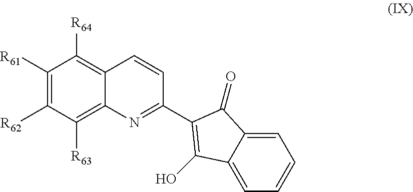

R₆₁ represents a hydrogen or halogen atom or an alkyl group;
R₆₂, R₆₃, and R₆₄, which may be identical or different, represent a hydrogen atom or a group (O)₂S(O⁻)—, M⁺ with M⁺ representing a hydrogen atom or a cationic counterion;

or alternatively R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;

it being understood that formula (IX) comprises at least one sulfonate radical (O)₂S(O⁻)—, M⁺ preferentially sodium sulfonate.

As examples of dyes of formula (IX), mention may be made of: Acid Yellow 2, Acid Yellow 3 and Acid Yellow 5.

More particularly, the dyes of formulae (II) to (VII) are chosen from:

| | |
|---|---|
| (C.I. 45380) | Acid Red 87 (VII) |
| (C.I. 10316) | Sodium salt of 2,4-dinitro-1-naphthol-7-sulfonic |
| | acid (V′) |
| (C.I. 10383) | Acid Orange 3 (V) |
| (C.I. 13015) | Acid Yellow 9/Food Yellow 2 (II) |
| (C.I. 14780) | Direct Red 45/Food Red 13 (II) |
| (C.I. 13711) | Acid Black 52 (II) |
| (C.I. 13065) | Acid Yellow 36 (II) |
| (C.I. 14700) | Sodium salt of 1-hydroxy-2-(2′,4′-xylyl-5- |
| | sulfonatoazo)naphthalene-4-sulfonic acid/Food |
| | Red 1 (II) |
| (C.I. 14720) | Acid Red 14/Food Red 3/Mordant Blue 79 (II) |
| (C.I. 14805) | Sodium salt of 4-hydroxy-3-[(2-methoxy-5- |
| | nitrophenyl)diaza]-6-(phenylamino)naphthalene- |
| | 2-sulfonic acid/Acid Brown 4 (II) |
| (C.I. 15510) | Acid Orange 7/Pigment Orange 17/Solvent |
| | Orange 49/Orange 4 (II) |
| (C.I. 15985) | Food Yellow 3/Pigment Yellow 104 (II) |
| (C.I. 16185) | Acid Red 27/Food Red 9 (II) |
| (C.I. 16230) | Acid Orange 10/Food Orange 4 (II) |
| (C.I. 16250) | Acid Red 44 (II) |
| (C.I. 17200) | Acid Red 33/Food Red 12 (II) |
| (C.I. 15685) | Acid Red 184 (II) |
| (C.I. 19125) | Acid Violet 3 (II) |
| (C.I. 18055) | Sodium salt of 1-hydroxy-2-(4′-acetamidophenyl- |
| | azo)-8-acetamidonaphthalene-3,6-disulfonic |
| | acid/Acid Violet 7/Food Red 11 (II) |
| (C.I. 18130) | Acid Red 135 (II) |
| (C.I. 19130) | Acid Yellow 27 (III) |
| (C.I. 19140) | Acid Yellow 23/Food Yellow 4 (III) |
| (C.I. 20170) | 4′-(Sulfonato-2″,4″-dimethyl)bis(2,6-phenylazo)- |
| | 1,3-dihydroxybenzene/Acid Orange 24 (II) |
| (C.I. 20470) | Sodium salt of 1-amino-2-(4′-nitrophenylazo)- |
| | 7-phenylazo-8-hydroxynaphthalene-3,6-disulfonic |
| | acid/Acid Black 1 (II) |
| (C.I. 23266) | (4-((4-Methylphenyl)sulfonyloxy)phenylazo)- |
| | 2,2′-dimethyl-4-((2-hydroxy-5,8-disulfonato)naphthylazo)- |
| | biphenyl/Acid Red 111 (II′) |
| (C.I. 27755) | Food Black 2 (II) |
| (C.I. 25440) | 1-(4′-Sulfonatophenylazo)-4-((2″-hydroxy-3″- |
| | acetylamino-6″,8″-disulfonato)naphthylazo)-6- |
| | sulfonatonaphthalene (tetrasodium salt)/Food Black 1 (II) |
| (C.I. 42090) | Acid Blue 9 (VI) |
| (C.I. 60730) | Acid Violet 43 (IV) |
| (C.I. 61570) | Acid Green 25 (IV) |
| (C.I. 62045) | Sodium salt of 1-amino-4-cyclohexylamino-9,10- |
| | anthraquinone-2-sulfonic acid/Acid Blue 62 (IV) |
| (C.I. 62105) | Acid Blue 78 (IV) |
| (C.I. 14710) | Sodium salt of 4-hydroxy-3-((2-methoxyphenyl)azo)- |
| | 1-naphthalenesulfonic acid/Acid Red 4 (II) |
| | 2-Piperidino-5-nitrobenzenesulfonic acid (V′) |
| | 2-(4′-N,N-(2″-Hydroxyethyl)amino-2′-nitro)ani- |
| | lineethanesulfonic acid (V′) |
| | 4-β-Hydroxyethylamino-3-nitrobenzenesulfonic acid (V′) |
| (C.I. 42640) | Acid Violet 49 (VI) |
| (C.I. 42080) | Acid Blue 7 (VI) |
| (C.I. 58005) | Sodium salt of 1,2-dihydroxy-3-sulfoanthraquinone/ |
| | Mordant Red 3 (IV) |
| (C.I. 62055) | Sodium salt of 1-amino-9,10-dihydro-9,10- |
| | dioxo-4-(phenylamino) 2-anthracenesulfonic |
| | acid/Acid Blue 25 (IV) |
| (C.I. 14710) | Sodium salt of 4-hydroxy-3-((2-methoxyphenyl)azo)- |
| | 1-naphthalenesulfonic acid/Acid Red 4 (II) |
| (C.I. 16255) | Acid Red 18 (II) |
| (C.I. 19140) | Yellow 5 (III) |

Most of these dyes are described in particular in the Colour Index published by The Society of Dyers and Colourists, P.O. Box 244, Perkin House, 82 Grattan Road, Bradford, Yorkshire, BD12 JBN England.

The anionic dyes that are most particularly preferred are the dyes designated in the Colour Index under the code:

C.I. 58005 (monosodium salt of 1,2-dihydroxy-9,10-anthraquinone-3-sulfonic acid),

C.I. 60730 (monosodium salt of 2-[(9,10-dihydro-4-hydroxy-9,10-dioxo-1-anthracenyl)amino]-5-methylbenzenesulfonic acid),

C.I. 15510 (monosodium salt of 4-[(2-hydroxy-1-naphthyl)azo]benzenesulfonic acid),

C.I. 15985 (disodium salt of 6-hydroxy-5-[(4-sulfophenyl)azo]-2-naphthalenesulfonic acid),

C.I. 17200 (disodium salt of 5-amino-4-hydroxy-3-(phenylazo)-2,7-naphthalenedisulfonic acid),

C.I. 20470 (disodium salt of 1-amino-2-(4′-nitrophenylazo)-7-phenylazo-8-hydroxy-3,6-naphthalenedisulfonic acid),

C.I. 42090 (disodium salt of N-ethyl-N-[4-[[4-[ethyl[3-sulfophenyl)methyl]amino]phenyl](2-sulfophenyl)methylene]-2,5-cyclohexadien-1-ylidene]-3-sulfobenzenemethanaminium hydroxide, inner salt),

C.I. 61570 (disodium salt of 2,2′-[(9,10-dihydro-9,10-dioxo-1,4-anthracenediyl)diimino]bis[5-methyl]benzenesulfonic acid),

C.I. 19140 (Yellow 5, the trisodium salt of 5-hydroxy-1-(4-sulfophenyl)-4-(4-sulfophenylazo)pyrazole-3-carboxylic acid),

C.I. 16255 (Acid Red 18, the trisodium salt of 7-hydroxy-8-[(4-sulfo-1-naphthalenyl)azo]-1,3-naphthalenedisulfonic acid).

Preferably, the anionic dyes are chosen from the compounds of formulae (II) and/or (III).

Use may also be made of compounds corresponding to the mesomeric or tautomeric forms of structures (II) to (IX).

The direct dye(s) (v) may be present in the composition according to the invention in a total content ranging from 0.001% to 15% by weight, preferably ranging from 0.005% to 10% by weight, preferentially ranging from 0.01% to 5% by weight, especially from 0.05% to 3% by weight, or even from 0.1% to 2% by weight, relative to the total weight of the composition.

In particular, the composition according to the invention preferably comprises the anionic direct dyes in a total content ranging from 0.001% to 15% by weight, preferably ranging from 0.005% to 10% by weight, preferentially ranging from 0.01% to 5% by weight, especially from 0.05% to 3% by weight, or even from 0.1% to 2% by weight, relative to the total weight of the composition.

Cationic Polymers

The cosmetic composition may also comprise one or more cationic polymers, preferably with a cationic charge density of greater than or equal to 4 milliequivalents/gram (meq/g).

The cationic charge density of a polymer corresponds to the number of moles of cationic charges per unit mass of polymer under conditions in which it is totally ionized. It may be determined by calculation if the structure of the polymer is known, i.e. the structure of the monomers constituting the polymer and their molar proportion or weight proportion. It may also be determined experimentally by the Kjeldahl method.

For the purposes of the present invention, the term “cationic polymer” denotes any non-silicone (not comprising any silicon atoms) polymer containing cationic groups and/or groups that can be ionized into cationic groups and not containing any anionic groups and/or groups that can be ionized into anionic groups.

The cationic polymers that may be used preferably have a weight-average molar mass (Mw) of between 500 and 5×10⁶ approximately and preferably between 10³ and 3×10⁶ approximately.

Among the cationic polymers, mention may be made more particularly of:

(1) homopolymers or copolymers derived from acrylic or methacrylic esters or amides and comprising at least one of the units of the following formulae:

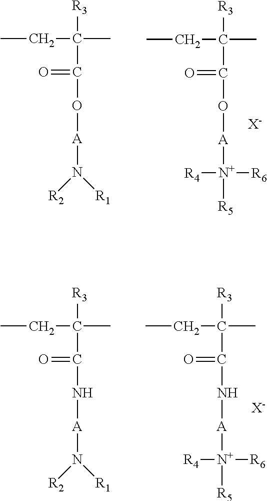

in which:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical, preferably an alkyl group containing from 1 to 6 carbon atoms;
R₁ and R₂, which may be identical or different, represent a hydrogen atom or an alkyl group containing from 1 to 6 carbon atoms, preferably methyl or ethyl;
X denotes an anion derived from a mineral or organic acid, such as a methosulfate anion or a halide such as chloride or bromide.

The copolymers of family (1) may also contain one or more units derived from comonomers that may be chosen from the family of acrylamides, methacrylamides, diacetone acrylamides, acrylamides and methacrylamides substituted on the nitrogen with lower (C1-C4) alkyls, acrylic or methacrylic acid esters, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as that sold under the name Hercofloc by the company Hercules,
copolymers of acrylamide and of methacryloyloxyethyltrimethylammonium chloride, such as the products sold under the name Bina Quat P 100 by the company Ciba Geigy,
the copolymer of acrylamide and of methacryloyloxyethyltrimethylammonium methosulfate, such as that sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937. These polymers are described in detail in French patents 2 077 143 and 2 393 573;
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the copolymers sold under the name Styleze CC 10 by ISP;
quaternized vinylpyrrolidone/dimethylaminopropylmethacrylamide copolymers such as the product sold under the name Gafquat HS 100 by the company ISP,
preferably crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.

(2) cationic polysaccharides, in particular cationic celluloses and galactomannan gums. Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are in particular described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble quaternary ammonium monomer are described in particular in U.S. Pat. No. 4,131,576, and mention may be made of hydroxyalkyl celluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by the company National Starch.

The cationic galactomannan gums are described more particularly in U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are in particular sold under the names Jaguar C13 S, Jaguar C 15, Jaguar C 17 and Jaguar C162 by the company Rhodia.

(3) polymers constituted of piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.

(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized.

(5) polyaminoamide derivatives resulting from the condensation of polyalkylene polyamines with polycarboxylic acids followed by alkylation with difunctional agents.

Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.

(6) polymers obtained by reacting a polyalkylene polyamine comprising two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or else under the name PD 170 or Delsette 101 by the company Hercules in the case of the adipic acid/epoxypropyl/diethylenetriamine copolymer.

(7) cyclopolymers of alkyldiallylamine or of dialkyldiallylammonium, such as the homopolymers or copolymers containing, as main constituent of the chain, units corresponding to formula (I) or (II):

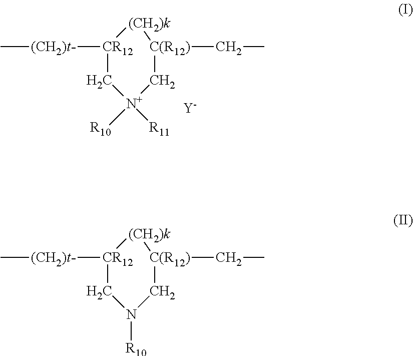

in which
k and t are equal to 0 or 1, the sum k+t being equal to 1;
R₁₂ denotes a hydrogen atom or a methyl radical;
R₁₀ and R₁₁, independently of each other, denote a C₁-C₆ alkyl group, a C₁-C₅ hydroxyalkyl group, a C₁-C₄ amidoalkyl group; or alternatively R₁₀ and R₁₁ may denote, together with the nitrogen atom to which they are attached, a heterocyclic group such as piperidyl or morpholinyl; R₁₀ and R₁₁, independently of each other, preferably denote a C₁-C₄ alkyl group;
Y⁻ is an anion such as bromide, chloride, acetate, borate, citrate, tartrate, bisulfate, bisulfite, sulfate or phosphate.

Mention may be made more particularly of the homopolymer of dimethyldiallylammonium salts (for example chloride) for example sold under the name Merquat 100 by the company Nalco and the copolymers of diallyldimethylammonium salts (for example chloride) and of acrylamide, sold in particular under the name Merquat 550 or Merquat 7SPR.

(8) quaternary diammonium polymers comprising repeating units of formula:

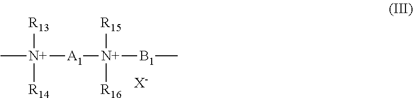

in which:
R₁₃, R₁₄, R₁₅ and R₁₆, which may be identical or different, represent aliphatic, alicyclic or arylaliphatic radicals comprising from 1 to 20 carbon atoms or C₁-C₁₂ hydroxyalkyl aliphatic radicals,

or else R₁₃, R₁₄, R₁₅ and R₁₆, together or separately, form, with the nitrogen atoms to which they are attached, heterocycles optionally comprising a second non-nitrogen heteroatom;

or else R₁₃, R₁₄, R₁₅ and R₁₆ represent a linear or branched C₁-C₆ alkyl radical substituted with a nitrile, ester, acyl, amide or —CO—O—R₁₇-D or —CO—NH—R₁₇-D group, where R₁₇ is an alkylene and D is a quaternary ammonium group;
A₁ and B₁ represent linear or branched, saturated or unsaturated, divalent polymethylene groups comprising from 2 to 20 carbon atoms, which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;

it being understood that A₁, R₁₃ and R₁₅ can form, with the two nitrogen atoms to which they are attached, a piperazine ring;

in addition, if A₁ denotes a linear or branched, saturated or unsaturated alkylene or hydroxyalkylene radical, B₁ may also denote a group (CH₂)n-CO-D-OC—(CH₂)p- with n and p, which may be identical or different, being integers ranging from 2 to 20, and D denoting:
a) a glycol residue of formula —O—Z—O—, in which Z denotes a linear or branched hydrocarbon-based radical, or a group corresponding to one of the following formulae: —(CH₂CH₂O)x-CH₂CH₂— and —[CH₂CH(CH₃)O]y-CH₂CH(CH₃)—, in which x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula —NH—Y—NH—, in which Y denotes a linear or branched hydrocarbon-based radical, or else the divalent radical —CH₂—CH₂—S—S—CH₂—CH₂—;
d) a ureylene group of formula —NH—CO—NH—.

Preferably, X⁻is an anion, such as chloride or bromide. These polymers have a number-average molar mass (Mn) generally of between 1000 and 100 000.

Mention may be made more particularly of polymers that are constituted of repeating units corresponding to the formula:

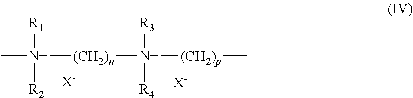

in which R₁, R₂, R₃ and R₄, which may be identical or different, denote an alkyl or hydroxyalkyl radical containing from 1 to 4 carbon atoms, n and p are integers ranging from 2 to 20, and X⁻is an anion derived from a mineral or organic acid.

A particularly preferred compound of formula (IV) is the one for which R₁, R₂, R₃ and R₄ represent a methyl radical and n=3, p=6 and X=Cl, known as Hexadimethrine chloride according to the INCI (CTFA) nomenclature.

(9) polyquaternary ammonium polymers comprising units of formula (V):

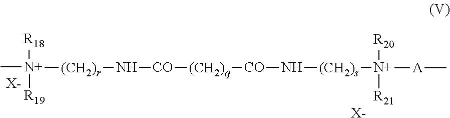

in which:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)pOH radical, in which p is equal to 0 or to an integer between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion such as a halide,
A denotes a divalent dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by the company Miranol.

(10) quaternary polymers of vinylpyrrolidone and of vinylimidazole, for instance the products sold under the names Luviquat® FC 905, FC 550 and FC 370 by the company BASF.

(11) polyamines such as Polyquart® H sold by Cognis, referred to under the name Polyethylene glycol (15) tallow polyamine in the CTFA dictionary.

(12) polymers comprising in their structure:

(a) one or more units corresponding to formula (A) below:

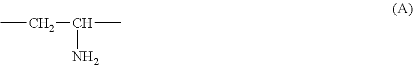

(b) optionally one or more units corresponding to formula (B) below:

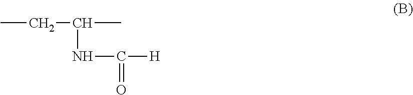

In other words, these polymers may be chosen in particular from homopolymers or copolymers comprising one or more units derived from vinylamine and optionally one or more units derived from vinylformamide.

Preferably, these cationic polymers are chosen from polymers comprising, in their structure, from 5 mol % to 100 mol % of units corresponding to the formula (A) and from 0 to 95 mol % of units corresponding to the formula (B), preferably from 10 mol % to 100 mol % of units corresponding to the formula (A) and from 0 to 90 mol % of units corresponding to the formula (B).

These polymers may be obtained, for example, by partial hydrolysis of polyvinylformamide. This hydrolysis may take place in acidic or basic medium.

The weight-average molecular weight of said polymer, measured by light scattering, may range from 1000 to 3 000 000 g/mol, preferably from 10 000 to 1 000 000 and more particularly from 100 000 to 500 000 g/mol.

The polymers comprising units of formula (A) and optionally units of formula (B) are sold in particular under the Lupamin name by the company BASF, for instance, in a non-limiting way, the products provided under the names Lupamin 9095, Lupamin 5095, Lupamin 1095, Lupamin 9030 (or Luviquat 9030) and Lupamin 9010.

Other cationic polymers that may be used in the context of the invention are cationic proteins or cationic protein hydrolysates, polyalkyleneimines, in particular polyethyleneimines, polymers comprising vinylpyridine or vinylpyridinium units, condensates of polyamines and of epichlorohydrin, quaternary polyureylenes and chitin derivatives.

Preferably, the cationic polymers are chosen from those of families (1), (2), (7) and (10) mentioned above.

Among the cationic polymers mentioned above, the ones that may preferably be used are cationic polysaccharides, in particular cationic celluloses and cationic galactomannan gums, and in particular quaternary cellulose ether derivatives such as the products sold under the name JR 400 by the company Amerchol, cationic cyclopolymers, in particular dimethyldiallylammonium salt (for example chloride) homopolymers or copolymers, sold under the names Merquat 100, Merquat 550 and Merquat S by the company Nalco, quaternary polymers of vinylpyrrolidone and of vinylimidazole, optionally crosslinked homopolymers or copolymers of methacryloyloxy(C1-C4)alkyltri(C1-C4)alkylammonium salts, and mixtures thereof.

The total content of cationic polymer(s) in the composition according to the invention may range from 0.05% to 5% by weight relative to the total weight of the composition, preferably from 0.1% to 3% by weight and preferentially from 0.2% to 2% by weight relative to the total weight of the composition.

Amphoteric Polymers

The cosmetic composition may also comprise one or more amphoteric polymers. For the purposes of the present invention, the term “amphoteric polymer” denotes any non-silicone (not comprising any silicon atoms) polymer containing cationic groups and/or groups that can be ionized into cationic groups and also anionic groups and/or groups that can be ionized into anionic groups.

The amphoteric polymers may preferably be chosen from amphoteric polymers comprising the repetition of:

(i) one or more units derived from a (meth)acrylamide-type monomer,

(ii) one or more units derived from a (meth)acrylamidoalkyltrialkylammonium-type monomer, and

(iii) one or more units derived from a (meth)acrylic acid-type acid monomer.

Preferably, the units derived from a (meth)acrylamide-type monomer are units of structure (Ia) below:

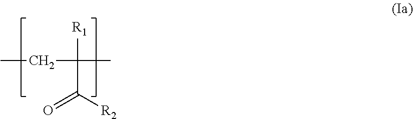

in which R₁ denotes H or CH₃ and R₂ is chosen from an amino, dimethylamino, tertbutylamino, dodecylamino and —NH—CH₂OH radical.

Preferably, said amphoteric polymer comprises the repetition of only one unit of formula (Ia).

The unit derived from a monomer of (meth)acrylamide type of formula (Ia) in which R₁ denotes H and R₂ is an amino radical (NH₂) is particularly preferred. It corresponds to the acrylamide monomer per se.

Preferably, the units derived from a monomer of (meth)acrylamidoalkyltrialkylammonium type are units of structure (IIa) below:

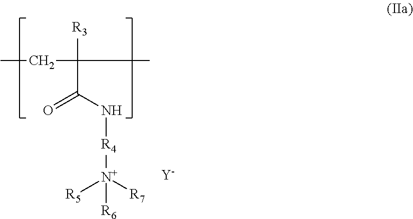

in which:
R₃ denotes H or CH₃,
R₄ denotes a group (CH₂)k, with k being an integer ranging from 1 to 6 and preferably from 2 to 4;
R₅, R₆ and R₇, which may be identical or different, denote a C₁-C₄ alkyl,
Y⁻is an anion such as bromide, chloride, acetate, borate, citrate, tartrate, bisulfate, bisulfite, sulfate or phosphate.

Preferably, said amphoteric polymer comprises the repetition of only one unit of formula (IIa).

Among these units derived from a (meth)acrylamidoalkyltrialkylammonium-type monomer of formula (IIa), the ones that are preferred are those derived from the methacrylamidopropyltrimethylammonium chloride monomer, for which R₃ denotes a methyl radical, k is equal to 3, R₅, R₆ and R₇ denote a methyl radical, and Y⁻ denotes a chloride anion.

Preferably, the units derived from a monomer of (meth)acrylic acid type are units of formula (IIIa):

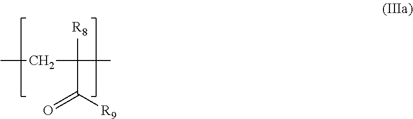

in which R₈ denotes H or CH₃ and R₉ denotes a hydroxyl radical or an —NH—C(CH₃)₂—CH₂—SO₃H radical.

The preferred units of formula (IIIa) correspond to the acrylic acid, methacrylic acid and 2-acrylamido-2-methylpropanesulfonic acid monomers.

Preferably, the unit derived from a monomer of (meth)acrylic acid type of formula (IIIa) is that derived from acrylic acid, for which R₈ denotes a hydrogen atom and R₉ denotes a hydroxyl radical.

The acidic monomer(s) of (meth)acrylic acid type may be non-neutralized or partially or totally neutralized with an organic or mineral base.

Preferably, said amphoteric polymer comprises the repetition of only one unit of formula (IIIa).

According to a preferred embodiment of the invention, the amphoteric polymer(s) of this type comprise at least 30 mol % of units derived from a monomer of (meth)acrylamide type (i). Preferably, they comprise from 30 mol % to 70 mol % and more preferably from 40 mol % to 60 mol % of units derived from a (meth)acrylamide-type monomer.

The content of units derived from a monomer of (meth)acrylamidoalkyltrialkylammonium type (ii) may advantageously be from 10 mol % to 60 mol % and preferentially from 20 mol % to 55 mol %.

The content of units derived from an acidic monomer of (meth)acrylic acid type (iii) may advantageously be from 1 mol % to 20 mol % and preferentially from 5 mol % to 15 mol %.

According to a particularly preferred embodiment of the invention, the amphoteric polymer of this type comprises:
from 30 mol % to 70 mol % and more preferably from 40 mol % to 60 mol % of units derived from a monomer of (meth)acrylamide type (i),
from 10 mol % to 60 mol % and preferentially from 20 mol % to 55 mol % of units derived from a monomer of (meth)acrylamidoalkyltrialkylammonium type (ii), and
from 1 mol % to 20 mol % and preferentially from 5 mol % to 15 mol % of units derived from a monomer of (meth)acrylic acid type (iii).

Amphoteric polymers of this type may also comprise additional units, other than the units derived from a (meth)acrylamide-type monomer, a (meth)acrylamidoalkyltrialkylammonium-type monomer and a (meth)acrylic acid-type monomer as described above.

However, according to a preferred embodiment of the invention, said amphoteric polymers are constituted solely of units derived from monomers of (meth)acrylamide type (i), of (meth)acrylamidoalkyltrialkylammonium type (ii) and of (meth)acrylic acid type (iii).

Mention may be made, as an example of particularly preferred amphoteric polymers, of acrylamide/methacrylamidopropyltrimethylammonium chloride/acrylic acid terpolymers. Such polymers are listed in the CTFA dictionary (INCI) under the name “Polyquaternium 53”. Corresponding products are in particular sold under the names Merquat 2003 and Merquat 2003 PR by Nalco.

As another type of amphoteric polymer that may be used, mention may also be made of copolymers based on (meth)acrylic acid and on a dialkyldiallylammonium salt, such as copolymers of (meth)acrylic acid and of dimethyldiallylammonium chloride. An example that may be mentioned is Merquat 280 sold by Nalco.

The amphoteric polymer(s) may generally be present in the composition according to the invention in a total amount of between 0.05% and 5% by weight, preferably between 0.1% and 3% by weight, and more particularly between 0.2% and 2% by weight, relative to the total weight of the composition.

According to a preferred embodiment of the invention, the composition according to the invention comprises:
one or more anionic surfactants (i) chosen from polyoxyalkylenated alkyl(amido)ether carboxylic acids and salts thereof, preferably of formula (I) as defined above;
one or more anionic surfactants (ii) chosen from sodium, triethanolamine, magnesium or ammonium (C12-C14)alkyl sulfates and/or sodium, ammonium or magnesium (C12-C14)alkyl ether sulfates, which are oxyethylenated, for example with 1 to 10 mol of ethylene oxide;
one or more nonionic surfactants in a total content of at least 1% by weight relative to the total weight of the composition, chosen, alone or as a mixture, from:
   saturated or unsaturated, linear or branched, oxyethylenated fatty alcohols comprising at least one C8 to C40, especially C8-C20 and better still C10-C18 alkyl chain and from 1 to 100 mol of ethylene oxide, preferably from 2 to 50, more particularly from 2 to 40 mol, or even from 3 to 20 mol of ethylene oxide and lauryl alcohol containing 12 mol of ethylene oxide; and
   (C6-C24 alkyl)(poly)glycosides, and more particularly (C8-C18 alkyl)(poly)glycosides;
one or more amphoteric surfactants, in a total content of at least 3% by weight relative to the total weight of the composition, comprising one or more surfactants chosen from (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines;
one or more direct dyes, preferably chosen, alone or as mixtures, from anionic direct dyes;
optionally, one or more cationic polymers preferably with a cationic charge density of greater than or equal to 4 meq/g, preferentially chosen from dialkyldiallylammonium halide homopolymers; and
optionally, one or more amphoteric polymers preferably constituted solely of units derived from monomers (i) of acrylamide type, (ii) of acrylamidoalkyltrialkylammonium type and (iii) of (meth)acrylic acid type.

The composition according to the invention may comprise water or a mixture of water and one or more cosmetically acceptable solvents chosen from Ci-C4 alcohols, such as ethanol, isopropanol, tert-butanol or n-butanol; polyols such as glycerol, propylene glycol and polyethylene glycols; and mixtures thereof.

Preferably, the composition according to the invention has a total water content of between 20% and 95% by weight, preferably between 30% and 90%, preferentially between 50% and 85% by weight and better still between 65% and 80% by weight relative to the total weight of the composition.

The pH of the compositions according to the invention generally ranges from 3 to 9, preferably from 3 to 7, preferentially from 3.5 to 6 and better still from 4 to 5.5.

The composition according to the invention may also comprise one or more standard additives that are well known in the art, such as natural or synthetic thickeners or viscosity regulators; C₁₂-C₃₀ fatty alcohols; ceramides; C₁₂-C₃₂ fatty esters such as isopropyl myristate, myristyl myristate, cetyl palmitate and stearyl stearate; mineral, plant or synthetic oils; vitamins or provitamins; nonionic or anionic polymers; pH stabilizers, preserving agents; dyes; fragrances; agents for preventing hair loss, anti-seborrhoeic agents, antidandruff agents.

A person skilled in the art will take care to select the optional additives and the amount thereof such that they do not harm the properties of the compositions of the present invention.

These additives are generally present in the composition according to the invention in an amount ranging from 0 to 20% by weight relative to the total weight of the composition.

The compositions in accordance with the invention may be used as shampoos for washing and conditioning the hair; they are preferably applied in this case to wet hair in amounts that are effective for washing it; the lather generated by massaging or rubbing with the hands may then be removed, after an optional leave-on time, by rinsing with water, the operation possibly being repeated one or more times.

Process and Use According to the Invention

Another subject of the present invention relates to a process for dyeing keratin fibres, in particular human keratin fibres such as the hair, comprising the application to said fibres of a composition as defined above, followed by an optional leave-on time and/or rinsing and/or drying.

The composition may be applied to wet or dry hair, and preferably to wet or moist hair.

According to one embodiment, the process consists in applying to keratin fibres an effective amount of the composition according to the invention, optionally massaging the fibres, optionally leaving the composition to stand on the fibres, and rinsing. The leave-on time of the composition on the keratin fibres may be between a few seconds and 30 minutes and preferably between 30 seconds and 20 minutes. The composition is generally rinsed out with water.

An optional step of drying the keratin fibres may be performed.

The present invention also relates to the use of the composition according to the invention as described previously for dyeing keratin fibres, in particular human keratin fibres such as the hair.

The examples that follow serve to illustrate the invention without, however, being limiting in nature.

### EXAMPLES

The cosmetic compositions according to the invention are prepared from the ingredients shown in the table below, the amounts of which are expressed as weight percentages of active material (AM).

Compositions (% AM)

| | A | B | C | D |
|---|---|---|---|---|
| Sodium Laureth Sulfate | 1.75 | 7 | 10 | 7 |
| Cocamidopropyl betaine | 3.8 | 7 | 7 | 7 |
| Laureth-5 carboxylic acid | 6.3 | 6 | 1 | 6 |
| Laureth-12 | 4 | 4 | 5.7 | — |
| Laureth-4 | 6 | 1 | 1 | — |
| Caprylyl/capryl glucoside | — | — | — | 3 |
| Polyquaternium-6 | 0.3 | 0.3 | 0.3 | 0.3 |
| Orange 4 | 0.2 | — | — | — |
| Yellow 5 | — | 0.2 | — | — |
| Acid Red 18 | — | — | 0.2 | 0.2 |
| Preserving agents, fragrance | qs | qs | qs | qs |
| pH agent | qs | qs | qs | qs |
| | pH 5 | pH 5 | pH 5 | pH 5 |
| Water | qs | qs | qs | qs |
| | 100% | 100% | 100% | 100% |

The compositions according to the invention may be used as shampoos.

1 g of composition was applied to 2.5 g locks of hair containing 90% white hairs, and the locks were then rinsed and dried. It was found that the locks thus treated were advantageously dyed, most particularly after several successive applications of the composition, especially after at least five successive applications. The hair has a very cosmetic feel: it is easy to disentangle and has a soft, smooth feel.

1.-18. (canceled)

19. A cosmetic composition, comprising:
(i) at least one anionic surfactant chosen from polyoxyalkylenated alkyl(amido)ether carboxylic acids or salts thereof;
(ii) at least one anionic surfactant other than the polyoxyalkylenated alkyl(amido)ether carboxylic acid anionic surfactants (i);
(iii) at least one nonionic surfactant chosen from:
(a) oxyethylenated alcohols comprising at least one saturated or unsaturated, linear or branched C8 to C40 alkyl chain, and comprising from 1 to 100 mol of ethylene oxide;
(b) nonionic surfactants of alkyl(poly)glycoside type, represented by the following general formula: R1O—(R2O)t-(G)v
wherein:
R1 represents a linear or branched alkyl or alkenyl radical comprising 6 to 24 carbon atoms, or an alkylphenyl radical whose linear or branched alkyl radical comprises 6 to 24 carbon atoms,
R2 represents an alkylene radical comprising 2 to 4 carbon atoms,
G represents a sugar unit comprising 5 to 6 carbon atoms,
t denotes a value ranging from 0 to 10,
v denotes a value ranging from 1 to 15; (c) polyethoxylated fatty acid esters of sorbitan;
(d) fatty acid esters of sucrose; or
(e) polyglycerolated fatty esters, wherein the number of glycerol groups ranges from 2 to 30 and comprising at least one saturated or unsaturated, linear or branched C8 to C40 alkyl chain.
(iv) at least one amphoteric surfactant; and
(v) at least one direct dye.

20. The composition according to claim 19, wherein the polyoxyalkylenated alkyl(amido)ether carboxylic acids are chosen from those of formula
R₁ [symbol] OC₂H₄)ₙ—OCH₂COOA (1)
in which:
R1 represents a linear or branched C6-C24 alkyl or alkenyl radical, a (C8-C9)alkylphenyl radical, a radical R2CONH—CH2-CH2- with R2 denoting a linear or branched C9-C21 alkyl or alkenyl radical;
n is an integer or decimal number ranging from 2 to 24,
A denotes H, ammonium, Na, K, Li, Mg, Ca, a monoethanolamine, or triethanolamine residue.

21. The composition according to claim 19, wherein the polyoxyalkylenated alkyl(amido)ether carboxylic acids are chosen from those of formula (1):
R₁ [symbol] OC₂H₄)ₙ—OCH₂COOA (1)
in which:
R1 denotes a C12-C14 alkyl, cocoyl, oleyl, nonylphenyl, or octylphenyl radical,
A denotes a hydrogen or sodium atom, and
n ranges from 2 to 20;

22. The composition of claim 21, wherein R1 denotes a C12 alkyl radical, A denotes a hydrogen or sodium atom, and n ranges from 2 to 10.

23. The composition according to claim 19, wherein the total amount of the at least one polyoxyalkylenated alkyl(amido)ether carboxylic acid/or salt thereof ranges from 0.05% to 30% by weight, relative to the total weight of the composition.

24. The composition according to claim 19, wherein the at least one anionic surfactant (ii) is chosen from:
C6-C24 alkyl sulfates;
C6-C24 alkyl ether sulfates;
C6-C24 alkylsulfosuccinates;
C6-C24 alkyl ether sulfosuccinates;
(C6-C24)acylisethionates;
C6-C24 C12-C20 acylsarcosinates;
C6-C24 acylglutamates; or
C6-C24 acylglycinates;
in the form of alkali metal or alkaline-earth metal, ammonium or amino alcohol salts.

25. The composition according to claim 19, wherein the at least one anionic surfactant (ii) is present in a total amount ranging from 0.05% to 30% by weight, relative to the total weight of the composition.

26. The composition according to claim 19, wherein the at least one nonionic surfactant is chosen from:
saturated or unsaturated, linear or branched, oxyethylenated C₈ to C₄₀₈ fatty alcohols comprising from 1 to 100 mol of ethylene oxide; or
(C6-C24 alkyl)(poly)glycosides.

27. The composition according to claim 19, wherein the at least one nonionic surfactant is present in a total amount ranging from 0.05% to 30% by weight, relative to the total weight of the composition.

28. The composition according to claim 19, wherein the at least one amphoteric surfactant is chosen from (C₈-C₂₀)alkylbetaines, (C₈-C₂₀)alkylsulfobetaines, (C₈-C₂₀)alkylamido(C₃-C₈)alkylbetaines, (C₈-C₂₀)alkylamido(C₆-C₈)alkylsulfobetaines, or
the compounds having the respective structures (II) and (III) below:
Rₐ—CONHCH₂CH₂—N⁺(R_{b})(R_{c})—CH₂COO⁻,M⁺,X⁻ (II)
wherein:
Rₐ represents a C₁₀ to C₃₀ alkyl or alkenyl group derived from an acid RₐCOOH present in hydrolysed coconut oil, a heptyl, nonyl, or undecyl group;
R_{b} represents a beta-hydroxyethyl group; and
R_{c} represents a carboxymethyl group;
M⁺ represents a cationic counterion derived from an alkali metal or alkaline-earth metal, an ammonium ion, or an ion derived from an organic amine; and
X⁻ represents an organic or mineral anionic counterion chosen from halides, acetates, phosphates, nitrates, (C₁-C₄)alkyl sulfates, (C₁-C₄)alkyl-, or (C₁-C₄)alkylarylsulfonates; or alternatively M⁺ and X⁻ are absent;
R_{a′}—CONHCH₂CH₂—N(B)(B′) (III)
wherein:
B represents —CH2CH2OX′;
B′ represents —(CH₂)_{z}Y′, with z=1 or 2;
X′ represents —CH₂COOH, —CH₂—COOZ′, CH₂CH₂COOH or —CH₂CH₂—COOZ′, or a hydrogen atom;
Y′ represents —COOH, —COOZ′, —CH₂—CH(OH)SO₃H, or CH₂CH(OH)SO₃—Z′;
Z′ represents a cationic counterion resulting from an alkali metal or alkaline-earth metal, an ammonium ion, or an ion resulting from an organic amine;
R_{a′} represents a C₁₀ to C₃₀ alkyl or alkenyl group of an acid R_{a′}—COOH which is present in coconut oil or in hydrolysed linseed oil, an alkyl group;
compounds of formula (IV):
R_{a″}—NHCH(Y″)—(CH₂)ₙCONH(CH₂)_{n′}—N(R_{d})(Rₑ) (IV)
wherein:
Y″ is —COOH, —COOZ″, —CH₂—CH(OH)SO₃H, or CH₂CH(OH)SO₃—Z″;
R_{d} and Rₑ represent, independently of each other, a C₁ to C₄ alkyl or hydroxyalkyl radical;
Z″ represents a cationic counterion derived from an alkali metal or alkaline-earth metal, an ammonium ion or an ion derived from an organic amine;
R_{a″} represents a C₁₀ to C₃₀ alkyl or alkenyl group of an acid R_{a″}—COOH which is present in coconut oil or in hydrolysed linseed oil;
n and n′ denote, independently of each other, an integer ranging from 1 to 3.

29. The composition according to claim 19, wherein the at least one amphoteric surfactant is present in a total amount ranging from 0.1% to 25% by weight, relative to the total weight of the composition.

30. The composition according to claim 19, wherein the at least one direct dye is chosen from synthetic or natural, anionic, cationic or nonionic direct dyes; from the anionic dyes of formulae (II), (II′), (III), (III′), (IV), (IV), (V), (V′), (VI), (VII), (VIII) and (IX) below:
a) the diaryl anionic azo dyes of formula (II) or (II′): 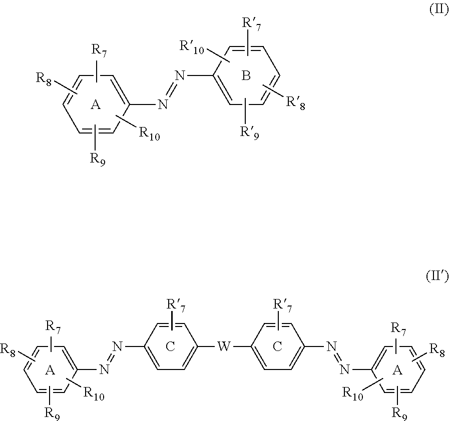
in which formulae (II) and (II′):
R₇, R₈, R₉, R₁₀, R′₇, R′₈, R′₉ and R′₁₀, which may be identical or different, represent a hydrogen atom or a group chosen from:
alkyl;
alkoxy, alkylthio;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ representing a hydrogen atom or a cationic counterion;
R″—S(O)₂—, with R″ representing a hydrogen atom or an alkyl, aryl, (di)(alkyl)amino, or aryl(alkyl)amino group;
R′″—S(O)₂—X′— with R′″ representing an alkyl or optionally substituted aryl group, X′ representing an oxygen or sulfur atom;
(di)(alkyl)amino;
aryl(alkyl)amino optionally substituted with one or more groups chosen from i) nitro; ii) nitroso; iii) (O)₂S(O⁻)—, M⁺ and iv) alkoxy with M⁺ representing a hydrogen atom or a cationic counterion;
optionally substituted heteroaryl; -cycloalkyl; or cyclohexyl;
Ar—N═N— with Ar representing an optionally substituted aryl group;
or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{∘}—C(X)—X′—; viii) R^{∘}—X′—C(X)—; ix) R^{∘}—X′—C(X)—X″—; x) Ar—N═N— and xi) optionally substituted aryl(alkyl)amino;
W represents a sigma bond, an oxygen or sulfur atom, or a divalent radical i) —NR—, or ii) methylene —C(Rₐ)(R_{b})— with Rₐ and R_{b}, which may be identical or different, representing a hydrogen atom or an aryl group, or alternatively Rₐ and R_{b} form, with the carbon atom that bears them, a spiro cycloalkyl;
wherein formulae (II) and (II′) comprise at least one sulfonate radical (O)₂S(O⁻)—, M⁺ or one carboxylate radical (O)CO⁻—, M⁺ on one of the rings A, A′, B, B′ or C;
b) the pyrazolone anionic azo dyes of formulae (III) and (III′): 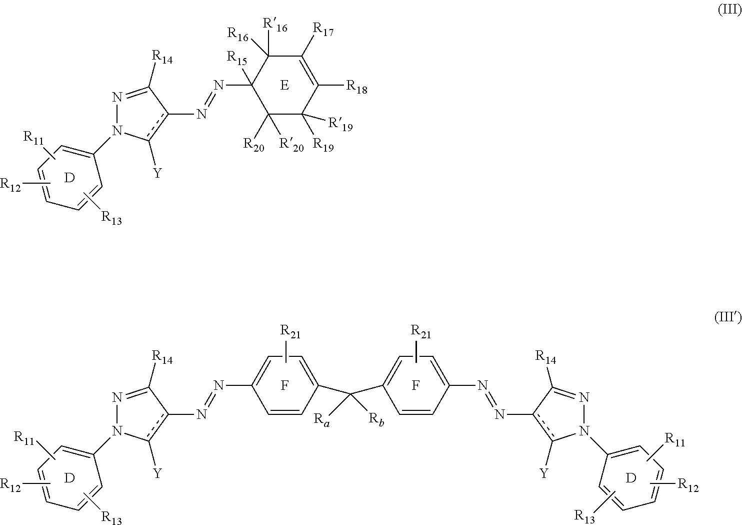
in which formulae (III) and (III′):
R₁₁, R₁₂ and R₁₃, which may be identical or different, represent a hydrogen or halogen atom, an alkyl group or —(O)₂S(O⁻), M⁺ with M⁺ representing a hydrogen atom or a cationic counterion;
R₁₄ represents a hydrogen atom, an alkyl group or a group —C(O)O—, M⁺ with M⁺ representing a hydrogen atom or a cationic counterion;
R₁₅ represents a hydrogen atom;
R₁₆ represents an oxo group, in which case R′₁₆ is absent, or alternatively R₁₅ with R₁₆ together form a double bond;
R₁₇ and R₁₈, which may be identical or different, represent a hydrogen atom, a group chosen from i) (O)₂S(O⁻)—, M⁺ with M⁺ representing a hydrogen atom or a cationic counterion, and ii) Ar—O—S(O)₂— with Ar representing an optionally substituted aryl group;
R₁₉ and R₂₀ together form either a double bond, or a benzo group D′, which is optionally substituted;
R′₁₆, R′₁₉ and R′₂₀, which may be identical or different, represent a hydrogen atom or an alkyl or hydroxyl group;
R₂₁ represents a hydrogen atom or an alkyl or alkoxy group;
Rₐ and R_{b}, which may be identical or different, representing a hydrogen atom or an aryl group;
Y represents either a hydroxyl group or an oxo group; [symbol]
represents a single bond when Y is an oxo group; and represents a double bond when Y represents a hydroxyl group;
wherein formulae (III) and (III′) comprise at least one sulfonate radical (O)₂S(O⁻)—, M⁺ or one carboxylate radical —C(O)O⁻—, M⁺ on one of the rings D or E;
c) the anthraquinone dyes of formulae (IV) and (IV′): 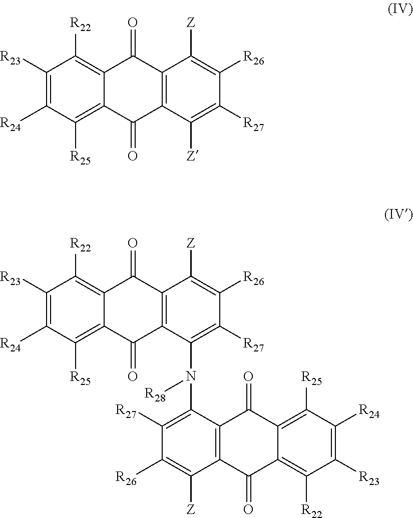
in which formulae (IV) and (IV′):
R₂₂, R₂₃, R₂₄, R₂₅, R₂₆ and R₂₇, which may be identical or different, represent a hydrogen or halogen atom, or a group chosen from:
alkyl;
hydroxyl, mercapto;
alkoxy, alkylthio;
optionally substituted aryloxy or arylthio;
aryl(alkyl)amino optionally substituted with one or more groups chosen from alkyl and (O)₂S(O⁻)—, M⁺ with M⁺ representing a hydrogen atom or a cationic counterion;
(di)(alkyl)amino;
(di)(hydroxyalkyl)amino;
(O)₂S(O⁻)—, M⁺ with M⁺ representing a hydrogen atom or a cationic counterion;
Z′ represents a hydrogen atom or a group NR₂₈R₂₉ with R₂₈ and R₂₉, which may be identical or different, representing a hydrogen atom or a group chosen from:
alkyl;
polyhydroxyalkyl such as hydroxyethyl;
aryl optionally substituted with one or more groups chosen from methyl, n-dodecyl, n-butyl; ii) (O)₂S(O—)—, M⁺; iii) R^{∘}—C(X)—X′—, R^{∘}—X′—C(X)—, R^{∘}—X′—C(X)—X″—, -cycloalkyl;
Z represents a group chosen from hydroxyl and NR′₂₈R′₂₉ wherein R′₂₈ and R′₂₉ may be identical or different;
wherein formulae (IV) and (IV′) comprise at least one sulfonate radical (O)₂S(O⁻)—, M⁺ or one carboxylate radical —C(O)O⁻—, M⁺;
d) the nitro dyes of formulae (V), (V′) and (V″): 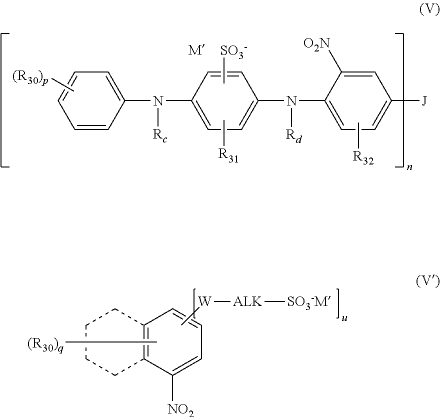
in which formulae (V) and (V′):
R₃₀, R₃₁ and R₃₂, which may be identical or different, represent a hydrogen or halogen atom, or a group chosen from:
alkyl;
alkoxy optionally substituted with one or more hydroxyl groups, alkylthio optionally substituted with one or more hydroxyl groups;
hydroxyl, mercapto;
nitro, nitroso;
polyhaloalkyl;
R^{∘}—C(X)—X′—, R^{∘}—X′—C(X)—, R^{∘}—X′—C(X)—X″—;
(O)₂S(O)—, M⁺;
(O)CO⁻—, M⁺;
(di)(alkyl)amino;
(di)(hydroxyalkyl)amino;
heterocycloalkyl such as piperidino, piperazino or morpholino;
in particular, R₃₀, R₃₁ and R₃₂ represent a hydrogen atom;
R_{c} and R_{d}, which may be identical or different, represent a hydrogen atom or an alkyl group;
W represents a group —NH—;
ALK represents a group —CH₂—CH₂—;
n is 1 or 2;
p represents an integer between 1 and 5 inclusive;
q represents an integer between 1 and 4 inclusive;
u is 0 or 1;
when n is 1, J represents a nitro or nitroso group;
when n is 2, J represents an oxygen or sulfur atom, or a divalent radical —S(O)ₘ— with m representing an integer 1 or 2;
M′ represents a hydrogen atom or a cationic counterion; 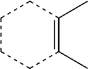
when it is present, represents a benzo group optionally substituted with one or more groups R₃₀;
it being understood that formulae (V) and (V′) comprise at least one sulfonate radical (O)₂S(O⁻)—, M⁺ or one carboxylate radical —C(O)O⁻—, M⁺;
e) the triarylmethane dyes of formula (VI): 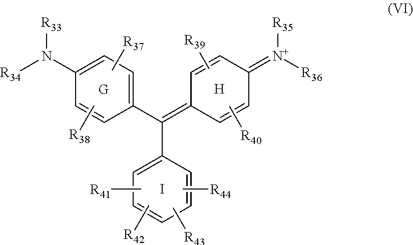
in which formula (VI):
R₃₃, R₃₄, R₃₅ and R₃₆, which may be identical or different, represent a hydrogen atom or a group chosen from alkyl, optionally substituted aryl and optionally substituted arylalkyl;
R₃₇, R₃₈, R₃₉, R₄₀, R₄₁, R₄₂, R₄₃ and R₄₄, which may be identical or different, represent a hydrogen atom or group chosen from:
alkyl;
alkoxy, alkylthio;
(di)(alkyl)amino;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺;
or alternatively two contiguous groups R₄₁ with R₄₂ or R₄₂ with R₄₃ or R₄₃ with R₄₄ together form a fused benzo group: I′; with I′ optionally substituted with one or more groups chosen from i) nitro; ii) nitroso; iii) (O)₂S(O⁻)—, M⁺; iv) hydroxyl; v) mercapto; vi) (di)(alkyl)amino; vii) R^{∘}—C(X)—X′—; viii) R^{∘}—X′—C(X)— and ix) R^{∘}—X′—C(X)—X″—;
wherein at least one of the rings G, H, I or I′ comprises at least one sulfonate radical (O)₂S(O⁻)— or a carboxylate radical —C(O)O⁻;
f) the xanthene-based dyes of formula (VII): 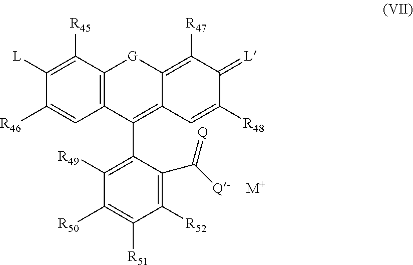
wherein:
R₄₅, R₄₆, R₄₇ and R₄₈, which may be identical or different, represent a hydrogen or halogen atom;
R₄₉, R₅₀, R₅₁ and R₅₂, which may be identical or different, represent a hydrogen or halogen atom, or a group chosen from:
alkyl;
alkoxy, alkylthio;
hydroxyl, mercapto;
nitro, nitroso;
(O)₂S(O⁻)—, M⁺ with M⁺ representing a hydrogen atom or a cationic counterion;
(O)CO⁻—, M⁺;
R₅₃, R₅₄, R₅₅ and R₄₈ represent a hydrogen or halogen atom;
G represents an oxygen or sulfur atom or a group NRₑ;
L represents an alkoxide O⁻, M⁺; a thioalkoxide S⁻, M⁺ or a group NR_{f}, with R_{f} representing a hydrogen atom or an alkyl group and M⁺ representing a hydrogen atom or a cationic counterion;
L′ represents an oxygen or sulfur atom or an ammonium group: N⁺R_{f}R_{g}, with R_{f} and R_{g}, which may be identical or different, representing a hydrogen atom, an alkyl group or optionally substituted aryl;
Q and Q′, which may be identical or different, represent an oxygen or sulfur atom;
M⁺ representing a hydrogen atom or a cationic counterion;
g) the indole-based dyes of formula (VIII): 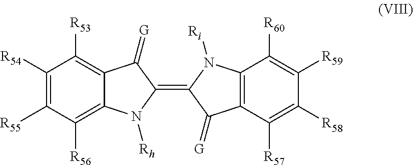
R₅₃, R₅₄, R₅₅, R₅₆, R₅₇, R₅₈, R₅₉ and R₆₀, which may be identical or different, represent a hydrogen atom or group chosen from:
alkyl;
alkoxy, alkylthio;
hydroxyl, mercapto;
nitro, nitroso;
R^{∘}—C(X)—X′—, R^{∘}—X′—C(X)—, R^{∘}—X′—C(X)—X″— with R^{∘} representing a hydrogen atom or an alkyl or aryl group; X, X′ and X″, which may be identical or different, representing an oxygen or sulfur atom, or NR with R representing a hydrogen atom or an alkyl group;
(O)₂S(O⁻)—, M⁺ with M⁺ representing a hydrogen atom or a cationic counterion;
(O)CO⁻—, M⁺ with M⁺ representing a hydrogen atom or a cationic counterion;
G represents an oxygen or sulfur atom or a group NRₑ with Rₑ;
Rᵢ and Rₕ, which may be identical or different, represent a hydrogen atom or an alkyl group;
wherein formula (VIII) comprises at least one sulfonate radical (O)₂S(O⁻)—, M⁺ or one carboxylate radical —C(O)O⁻, M⁺;
h) the quinoline-based dyes of formula (IX): 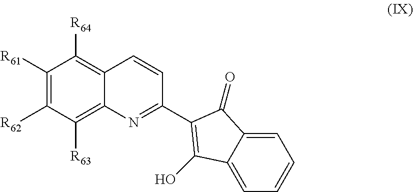
R₆₁ represents a hydrogen or halogen atom or an alkyl group;
R₆₂, R₆₃, and R₆₄, which may be identical or different, represent a hydrogen atom or a group (O)₂S(O⁻)—, M⁺ with M⁺ representing a hydrogen atom or a cationic counterion;
or alternatively R₆₁ with R₆₂, or R₆₁ with R₆₄, together form a benzo group optionally substituted with one or more groups (O)₂S(O⁻)—, M⁺ with M⁺ representing a hydrogen atom or a cationic counterion;
wherein formula (IX) comprises at least one sulfonate radical (O)₂S(O⁻)—.

31. The composition according to claim 19, wherein the at least one direct dye is chosen from the following dyes:

| | |
|---|---|
| (C.I. 45380) | Acid Red 87 (VII) |
| (C.I. 10316) | Sodium salt of 2,4-dinitro-1-naphthol-7-sulfonic |
| | acid (V′) |
| (C.I. 10383) | Acid Orange 3 (V) |
| (C.I. 13015) | Acid Yellow 9/Food Yellow 2 (II) |
| (C.I. 14780) | Direct Red 45/Food Red 13 (II) |
| (C.I. 13711) | Acid Black 52 (II) |
| (C.I. 13065) | Acid Yellow 36 (II) |
| (C.I. 14700) | Sodium salt of 1-hydroxy-2-(2′,4′-xylyl-5- |
| | sulfonatoazo)naphthalene-4-sulfonic acid/Food |
| | Red 1 (II) |
| (C.I. 14720) | Acid Red 14/Food Red 3/Mordant Blue 79 (II) |
| (C.I. 14805) | Sodium salt of 4-hydroxy-3-[(2-methoxy-5- |
| | nitrophenyl)diaza]-6-(phenylamino)naphthalene- |
| | 2-sulfonic acid/Acid Brown 4 (II) |
| (C.I. 15510) | Acid Orange 7/Pigment Orange 17/Solvent |
| | Orange 49/Orange 4 (II) |
| (C.I. 15985) | Food Yellow 3/Pigment Yellow 104 (II) |
| (C.I. 16185) | Acid Red 27/Food Red 9 (II) |
| (C.I. 16230) | Acid Orange 10/Food Orange 4 (II) |
| (C.I. 16250) | Acid Red 44 (II) |
| (C.I. 17200) | Acid Red 33/Food Red 12 (II) |
| (C.I. 15685) | Acid Red 184 (II) |
| (C.I. 19125) | Acid Violet 3 (II) |
| (C.I. 18055) | Sodium salt of 1-hydroxy-2-(4′-acetamido- |
| | phenylazo)-8-acetamidonaphthalene-3,6-disulfonic |
| | acid/Acid Violet 7/Food Red 11 (II) |
| (C.I. 18130) | Acid Red 135 (II) |
| (C.I. 19130) | Acid Yellow 27(III) |
| (C.I. 19140) | Acid Yellow 23/Food Yellow 4 (III) |
| (C.I. 20170) | 4′-(Sulfonato-2″,4″-dimethyl)bis(2,6-phenylazo)-1,3- |
| | dihydroxybenzene/Acid Orange 24 (II) |
| (C.I. 20470) | Sodium salt of 1-amino-2-(4′-nitrophenylazo)- |
| | 7-phenylazo-8-hydroxynaphthalene-3,6-disulfonic |
| | acid/Acid Black 1 (II) |
| (C.I. 23266) | (4-((4-Methylphenyl)sulfonyloxy)phenylazo)- |
| | 2,2′-dimethyl-4-((2-hydroxy-5,8-disulfonato)- |
| | naphthylazo)biphenyl/Acid Red 111 (II′) |
| (C.I. 27755) | Food Black 2 (II) |
| (C.I. 25440) | 1-(4′-Sulfonatophenylazo)-4-((2″-hydroxy-3″- |
| | acetylamino-6″,8″-disulfonato)naphthylazo)- |
| | 6-sulfonatonaphthalene (tetrasodium salt)/Food |
| | Black 1 (II) |
| (C.I. 42090) | Acid Blue 9 (VI) |
| (C.I. 60730) | Acid Violet 43 (IV) |
| (C.I. 61570) | Acid Green 25 (IV) |
| (C.I. 62045) | Sodium salt of 1-amino-4-cyclohexylamino-9,10- |
| | anthraquinone-2-sulfonic acid/Acid Blue 62 (IV) |
| (C.I. 62105) | Acid Blue 78 (IV) |
| (C.I. 14710) | Sodium salt of 4-hydroxy-3-((2-methoxyphenyl)azo)- |
| | 1-naphthalenesulfonic acid/Acid Red 4 (II) |
| | 2-Piperidino-5-nitrobenzenesulfonic acid (V′) |
| | 2-(4′-N,N-(2″-Hydroxyethyl)amino-2′-nitro)ani- |
| | lineethanesulfonic acid (V′) |
| | 4-β-Hydroxyethylamino-3-nitrobenzenesulfonic |
| | acid (V′) |
| (C.I. 42640) | Acid Violet 49 (VI) |
| (C.I. 42080) | Acid Blue 7 (VI) |
| (C.I. 58005) | Sodium salt of 1,2-dihydroxy-3-sulfoanthraquinone/ |
| | Mordant Red 3 (IV) |
| (C.I. 62055) | Sodium salt of 1-amino-9,10-dihydro-9,10-dioxo-4- |
| | (phenylamino) 2-anthracenesulfonic acid/Acid Blue 25 (IV) |
| (C.I. 14710) | Sodium salt of 4-hydroxy-3-((2-methoxyphenyl)azo)- |
| | 1-naphthalenesulfonic acid/Acid Red 4 (II) |
| (C.I. 16255) | Acid Red 18 (II) |
| (C.I. 19140) | Yellow 5 (III) |

32. The composition according to claim 19, wherein the at least one direct dye is present in a total amount ranging from 0.001% to 15% by weight, relative to the total weight of the composition.

33. The composition according to claim 19, further comprising at least one cationic polymer with a cationic charge density of greater than or equal to 4 milliequivalents/gram (meq/g); in a total amount ranging from 0.05% to 5% by weight, relative to the total weight of the composition.

34. The composition according to claim 19, further comprising at least one amphoteric polymer chosen from amphoteric polymers comprising the repetition of:
(i) one or more units derived from a (meth)acrylamide-type monomer,
(ii) one or more units derived from a (meth)acrylamidoalkyltrialkylammonium-type monomer, or
(iii) one or more units derived from a (meth)acrylic acid-type acid monomer;
wherein the at least one amphoteric polymer is present in a total amount of between 0.05% and 5% by weight, relative to the total weight of the composition.

35. The composition according to claim 19, further comprising water in a total amount of between 20% and 95% by weight, relative to the total weight of the composition.

36. A method for dyeing keratin fibers, comprising applying to said fibers a composition comprising:
(i) at least one anionic surfactant chosen from polyoxyalkylenated alkyl(amido)ether carboxylic acids or salts thereof;
(ii) at least one anionic surfactant other than the polyoxyalkylenated alkyl(amido)ether carboxylic acid anionic surfactants (i);
(iii) at least one nonionic surfactant chosen from:
(a) oxyethylenated alcohols comprising at least one saturated or unsaturated, linear or branched C8 to C40 alkyl chain, and comprising from 1 to 100 mol of ethylene oxide;
(b) nonionic surfactants of alkyl(poly)glycoside type, represented by the following general formula: R1O—(R2O)t-(G)v
wherein:
R1 represents a linear or branched alkyl or alkenyl radical comprising 6 to 24 carbon atoms, or an alkylphenyl radical whose linear or branched alkyl radical comprises 6 to 24 carbon atoms,
R2 represents an alkylene radical comprising 2 to 4 carbon atoms,
G represents a sugar unit comprising 5 to 6 carbon atoms,
t denotes a value ranging from 0 to 10,
v denotes a value ranging from 1 to 15;
(c) polyethoxylated fatty acid esters of sorbitan;
(d) fatty acid esters of sucrose; or
(e) polyglycerolated fatty esters, wherein the number of glycerol groups ranges from 2 to 30 and comprising at least one saturated or unsaturated, linear or branched C8 to C40 alkyl chain.
(iv) at least one amphoteric surfactant; and
(v) at least one direct dye.
